(19) United States
(12) Patent Application Publication
(10) Pub. No.: US 2021/0007304 A1
(43) Pub. Date: Jan. 14, 2021

(54) GROWING SYSTEM AND METHOD

(71) Applicant: Silo Farms, LLC, Springfield, MO (US)

(72) Inventor: Bradford Kuhns, Ontario (CA)

(21) Appl. No.: 17/041,829

(22) PCT Filed: Mar. 26, 2019
(86) PCT No.: PCT/US19/24008

§ 371 Date: Sep. 25, 2020

Related U.S. Application Data
(60) Provisional application No. 62/648,032, filed on Mar. 26, 2018.

(51) Int. Cl. A01G 31/04; E04H 7/22; A01G 7/04; A01G 18/69; A01G 31/06; A01G 18/62

(52) U.S. Cl. CPC A01G 31/042; E04H 7/22; A01G 18/62; A01G 18/69; A01G 31/06; A01G 7/045

(57) ABSTRACT

A spiral growing system is configured to be housed in a vertically elongated silo growth chamber to grow an agricultural crop. The spiral growing system broadly includes a spiral growing assembly and a movable crop support. The spiral growing assembly extends along an assembly length to at least partly define a spiral growing space to receive and feed the agricultural crop therein. The spiral growing assembly includes a continuous track and a feeding system. The track extends continuously along the assembly length and is configured to direct the agricultural crop along a generally downward spiral path. The movable crop support is operably supported by the track and is configured to be advanced downwardly along the assembly length to thereby direct the agricultural crop through the growing space with the feeding system providing direct root application of a supply of water and/or nutrients to the agricultural crop along the track.

### CROSS-REFERENCE TO RELATED APPLICATIONS

The present application claims the priority benefit of U.S. Provisional Patent Application Ser. No. 62/648,032, filed Mar. 26, 2018, entitled GROWING SYSTEM AND METHOD, which is hereby incorporated by reference in its entirety herein.

### BACKGROUND

### Field

The present invention relates generally to crop growing systems using direct root application of water and/or nutrients. Embodiments of the present invention concern a spiral growing assembly installed in a silo and configured for growing batches of agricultural crops using aeroponics, fogponics, Nutrient Film Technique, and/or related direct root application techniques.

### Description of Related Art

Plant growing systems utilizing direct root application of water and nutrients are well known in the art. Conventional systems are known to utilize hydroponic, aquaculture, and/or aquaponic techniques to grow various types of plants and animals. Known hydroponic systems include aeroponic and fogponic systems that use a spraying or misting system to feed plants.

However, conventional hydroponic, aeroponic, and fogponic systems all have various deficiencies. For instance, these conventional systems are incapable of producing vegetable and other plant products on an industrial scale consistent with modern/current farming and are generally not economically viable for large scale crop production. Known aeroponic and fogponics systems are particularly inefficient with respect to crop production speed and lack the needed throughput to be economically sustainable. Known systems are also inefficient concerning water usage and energy usage (both electrical and thermal). Furthermore, conventional systems are inadequately designed to maximize worker productivity (both in the number of workers and worker efficiency) and available facility space.

### SUMMARY

The following brief summary is provided to indicate the nature of the subject matter disclosed herein. While certain aspects of the present invention are described below, the summary is not intended to limit the scope of the present invention.

Embodiments of the present invention provide a spiral growing system that does not suffer from the problems and limitations of the prior art plant growing systems set forth above.

A first aspect of the present invention concerns a silo growing system configured to grow an agricultural crop. The silo growing system broadly includes a silo, a spiral growing assembly, and a movable crop support. The silo presents a vertically elongated silo growth chamber. The spiral growing assembly is positioned in the silo growth chamber and extends along an assembly length to at least partly define a spiral growing space to receive and feed the agricultural crop therein. The spiral growing assembly includes a continuous track and a feeding system. The track extends continuously along the assembly length of the spiral growing assembly and presents a generally downward spiral path that defines a path axis, with the track configured to direct the agricultural crop along the spiral path. The movable crop support is configured to support at least some of the agricultural crop. The movable crop support is operably supported by the track and is configured to be advanced downwardly along the assembly length to thereby direct the agricultural crop through the growing space along the spiral path. The feeding system extends along the track to direct a supply of water and/or nutrients in the growing space along the spiral path by providing direct root application of the supply of water and/or nutrients to the agricultural crop.

A second aspect of the present invention concerns a spiral growing system configured to be housed in a vertically elongated silo growth chamber to grow an agricultural crop. The spiral growing system broadly includes a spiral growing assembly and a movable crop support. The spiral growing assembly extends along an assembly length to at least partly define a spiral growing space to receive and feed the agricultural crop therein. The spiral growing assembly includes a continuous track and a feeding system. The track extends continuously along the assembly length of the spiral growing assembly and presents a generally downward spiral path that defines a path axis, with the track configured to direct the agricultural crop along the spiral path. The movable crop support is configured to support at least some of the agricultural crop. The movable crop support is operably supported by the track and is configured to be advanced downwardly along the assembly length to thereby direct the agricultural crop through the growing space along the spiral path. The feeding system extends along the track to direct a supply of water and/or nutrients in the growing space along the spiral path by providing direct root application of the supply of water and/or nutrients to the agricultural crop.

A third aspect of the present invention concerns a method of growing an agricultural crop using aeroponics, fogponics, and/or nutrient film technique. The method includes the steps of positioning agricultural crop on a spiral path; facilitating advancement of the agricultural crop downwardly along the spiral path; providing direct root application of water and/or nutrients to the agricultural crop to grow the agricultural crop as the agricultural crop are advanced along the spiral path; and harvesting the agricultural crop from the spiral path.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWINGS

Preferred embodiments of the invention are described in detail below with reference to the attached drawing figures, wherein:

FIG. 1 is a perspective of a silo growing system constructed in accordance with a preferred embodiment of the present invention, showing a silo building with a plurality of silos and a plurality of spiral growing assemblies installed in the silos, with sections of silo walls removed to depict a schematic representation of the spiral growing assemblies;

FIG. 2 is an enlarged fragmentary perspective of the silo growing system shown in FIG. 1, with the spiral growing assemblies being depicted schematically;

FIG. 3 is a top plan view of the silo growing system shown in FIGS. 1 and 2, showing the system cross-sectioned to depict the spiral growing assemblies in the silos and interstitial vertical bins located between the silos;

FIG. 4 is a fragmentary upper perspective of one of the silos and spiral growing assemblies shown in FIGS. 1-3, showing a spiral segment of the growing assembly supporting a train of movable carts, with the growing assembly including a continuous spiral track, a feeding system, a lighting system, and an air system;

FIG. 5 is a fragmentary lower perspective of the silo, spiral growing assembly, and train similar to FIG. 4, but showing an underneath view of the components, with the feeding system including a spiral bedway underneath the movable carts and the lighting system including LED lights mounted underneath the spiral bedway;

FIG. 6 is a fragmentary top plan view of the silo, spiral growing assembly, and train shown in FIGS. 4 and 5;

FIG. 7 is a fragmentary top plan view of the silo growing system similar to FIG. 3, but showing bin chambers presented by one of the interstitial bins, with the bin chambers having cooling air supplied by fan units of the air system and distributing the cooling air to air ducts installed in adjacent silos;

FIG. 7A is an enlarged fragmentary top plan view of the silo growing system similar to FIG. 7, but showing the arrangement of air ducts in one of the silos, with each air duct being supplied with cooling air via respective air inlet openings presented by the silo wall, and further depicting a circumferential flow of cooling air along the air ducts and a radially inward flow of cooling air streams discharged out of the air ducts;

FIG. 8 is a fragmentary upper perspective of the bedway and LED lights shown in FIGS. 4 and 5, showing a collection tray of the bedway, with the tray having opposite sidewalls and a bottom wall, and further showing water and nutrient nozzles mounted to extend through the inner sidewall;

FIG. 9 is a fragmentary lower perspective of the bedway and LED lights similar to FIG. 8, but showing an underneath view that depicts the LED lights spaced along the tray;

FIG. 10 is a fragmentary upper perspective of the bedway similar to FIG. 8, and further showing a gutter and downspout associated with the collection tray to collect excess water and/or nutrients;

FIG. 11 is a fragmentary perspective of the silo, spiral growing assembly, and train shown in FIGS. 4-6, showing part of one air duct broken away to depict an air inlet opening that supplies cooling air from the interstitial bin to the air duct, and further showing an outlet of the air duct comprising a pattern of holes spaced along the air duct;

FIG. 12 is an enlarged fragmentary perspective of the air duct shown in FIG. 11, showing one of the ends of the air duct enclosed and depicting the pattern of holes presented along the circumferential face of the air duct;

FIG. 13 is a schematic view of the spiral growing assembly shown in FIGS. 1-3, showing a controller, a water system, a nutrient system, and a liquid return system of the feeding system;

FIG. 14 is a schematic cross section of one spiral growing assembly shown in FIGS. 1-3, showing movable carts positioned on multiple levels of the track above respective parts of the bedway, with each movable cart including a frame, wheels, and a cap to receive plants, and showing a spacing between adjacent levels progressively increasing in the downward direction to accommodate plant growth;

FIG. 14A is an enlarged fragmentary cross section of the one spiral growing assembly similar to FIG. 14, and showing the movable cart located above the bedway to define a feed zone therebetween for spraying/misting the plant roots, and further showing the movable cart located below LED lights to define a lighting zone therebetween for illuminating the plant canopy to facilitate plant photosynthesis;

FIG. 15 is another enlarged fragmentary cross section of the one spiral growing assembly similar to FIG. 14, showing a movable control cart of the train received on the track, with the control cart including a braking mechanism and cleaning device shown schematically, where the braking mechanism is configured to control advancement of the train and the cleaning device is operable to clean the bedway during advancement;

FIG. 16 is a side elevation of the one spiral growing assembly shown in FIG. 15, showing a cleaning tank with cleaning solution, spray nozzles, a squeegee, and a rotating brush of the cleaning device; and

FIG. 17 is a schematic cross section of another spiral growing assembly shown in FIGS. 1-3, showing movable carts received on multiple levels of the track, with each movable cart including a frame and wheels to receive mushroom bags for incubation, and showing a constant spacing between adjacent levels of the track.

The drawing figures do not limit the present invention to the specific embodiments disclosed and described herein. The drawings are not necessarily to scale, emphasis instead being placed upon clearly illustrating the principles of the preferred embodiment.

### DETAILED DESCRIPTION

Turning initially to FIGS. 1-3, 14, and 14A, a silo growing system 30 provides a production facility for growing various crops of plants P using direct root application of water and/or nutrients.

The illustrated silo growing system 30 includes multiple spiral growing assemblies 32 that each preferably have a vertical spiral (i.e., helix) orientation. The silo growing system 30 utilizes a number of energy efficiency systems and improvements to maximize the output of the system and minimize the energy necessary for the operation of the system.

In particular, each spiral growing assembly 32 is configured as a helix mounted in a silo growing chamber and arranged on a vertically-oriented central access shaft T running along the axis of the helix. In this manner, a plurality of plant supports can be arranged in succession following the helical path of the vertically elongated spiral track, winding downward around the central access shaft T. It will be appreciated that the depicted silo growing system is configured as a helix or coil with a constant diameter, in contrast to a horizontal greenhouse conveyor system.

As will be described in greater detail, the silo growing system 30 broadly includes spiral growing assemblies 32, a silo building 34, and a plurality of movable crop supports 36.

Various crops of agricultural products can be grown using this system and may include plants (such as plants P), fungi (such as mushrooms provided in mushroom bags M), and/or animals. Such agricultural crops can include, without limitation, food crops (for human consumption), feed crops (for animal consumption), fiber crops (for textiles), oil crops (biodiesel), and industrial crops (pharmaceutical, cosmetic), such as lettuces, leafy greens, melons, berries, grapes, cannabis, herbs, fungi (e.g., mushrooms), and the like. Thus, the plants P may include vegetables, fruits, grains, etc. The mushroom bags M may include various types of mushrooms or other fungi. Although the illustrated embodiment depicts the use of system 30 to grow plants P and incubate mushrooms, it is entirely within the ambit of the present invention where the system 30 is configured to grow crops of other agricultural products.

As used herein, the term “fungi” generally includes, without limitation, mycelium, spawn, mushrooms, and similar terms. It will be understood that such fungi may be provided in the depicted bags, other types of fungi bags, or various other containers for fungi, or may be otherwise carried/supported by support structure suitable for fungi incubation. Furthermore, fungi may or may not be provided with various substrates, such as sawdust, straw, or other materials. It will be understood that a substrate can be colonized with fungi to provide various end products (e.g., fungi intended for mushroom fruit body production or simply a colonized substrate). For instance, it is within the scope of the present invention where a plant substrate (e.g., millet, sorghum, or rice) is colonized with fungi. The colonized substrate can then be dried and ground into a nutrified medicinal product or a nutritionally enhanced flour. The colonized substrate could also serve as spawn for later use (e.g., by other growers). The mushroom bags M preferably include mushrooms but could also include various types of fungi and may be referred to as a fungi bag.

Again, it is also within the ambit of the present invention where the silo growing system is used to grow animals. For instance, the system may be configured to grow crickets (or other insects), worms, larvae, etc. In some embodiments, it will be understood that combinations of crops can be provided together as part of an end product. For example, insects can be used as a protein source in the mushroom mix (e.g., where the insects are ground up and pelletized into the combo pellets).

Growth cycles of crops may range from about twelve (12) days to about sixty (60) days, with the growth cycle being completed for harvesting when the plants reach the bottom of the spiral. Thus, the speed of travel of the crop supports 36 can be adjusted accordingly. Further, the speed of travel may be constant (e.g., 2-inches per hour) or may involve periods of stop/start throughout the growth cycle.

### Direct Root Application Techniques

The devices, systems, and methods disclosed herein relate to a crop growing system and methods that provide direct root application of water and/or nutrients to plants P (and/or other agricultural crops). Such direct root application preferably includes direct root spraying systems (e.g., aeroponics, fogponics, etc.). For certain aspects of the present invention, water and/or nutrients could be applied using other direct root application methods, such as nutrient film technique (NFT).

In preferred embodiments, the disclosed system 30 is operable to provide an air/mist environment, utilizing direct root spraying systems. As will be described, crops of plants P (and/or other agricultural crops) are grown suspended in/on crop supports 36 that travel through the growth chamber from the beginning of the growth cycle (top) to harvesting (bottom). In the case of vegetables, and other agricultural crops, the crop supports 36 are configured so that the body, leaves, and/or crown of the plant (i.e., canopy C) are separated from roots R by the crop support 36. Instead of being rooted in a growth medium or immersed in water, the roots R hang free and are exposed to the ambient environment (air) of the growth chamber in the space between the crop support structure and the drip tray. When needed, water (moisture) and nutrients are delivered directly (and in most cases exclusively) to the crop's dangling roots R and lower stem that extend below the crop support structure via an atomized or sprayed nutrient-rich water solution. The rest of the plant (e.g., leaves, etc.) remains relatively dry.

Depending upon the nozzles used to generate the spray, the water and/or nutrient solution delivered to the roots may be the form of macro water droplets, micro-droplets of mist, or even smaller fog droplets. The crops are exposed to a light source (e.g., artificial light) at appropriate intervals for the crop type as the crop support structure travels along the inventive crop conveyor system.

As will also be discussed, the silo growing system 30 includes a feeding and lighting configuration that provides a desired series of crop growth cycles. The growth cycles are generally associated with advancement of crops downwardly along a spiral path.

Preferably, the plants P (and/or other agricultural crops) are fed with water and nutrients during particular feeding intervals associated with a growth cycle. Additionally, the plants P may also be fed during intermediate intervals (e.g., intermediate rest intervals). That is, each pair of adjacent feeding intervals is preferably separated by an intermediate interval. The plants P may be fed using a modified feeding schedule or varied nutrient levels during the intermediate intervals.

### Silo Facility

The illustrated silo building 34 houses the spiral growing assemblies 32 and movable crop supports 36. The silo building 34 preferably comprises a conventional grain elevator that includes a plurality of vertical silos 38a,b arranged alongside one another. The silo building 34 also preferably includes a basement (not shown) that extends laterally beneath the silos 38 and a gallery 40 that extends laterally along the top of the silos 38.

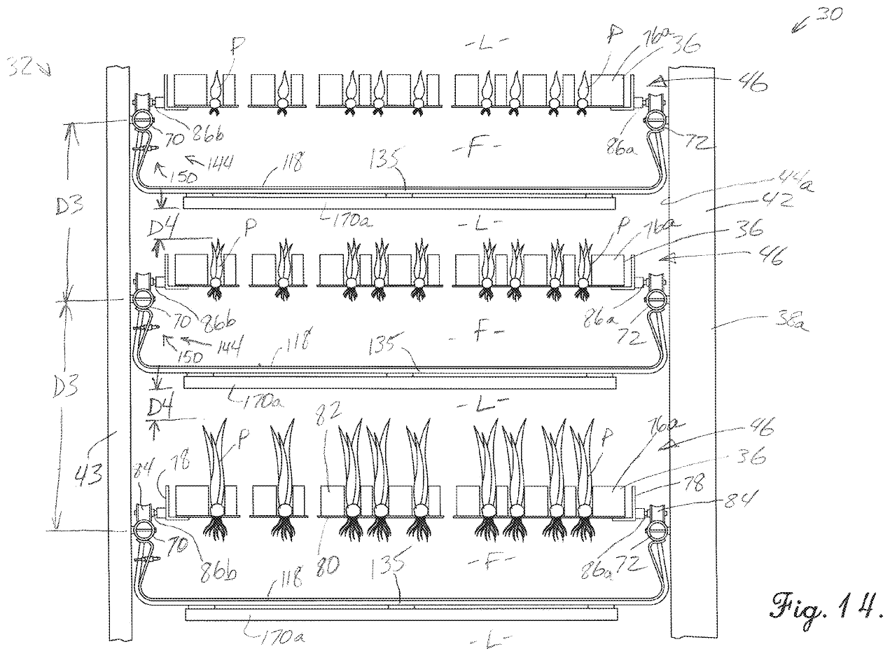

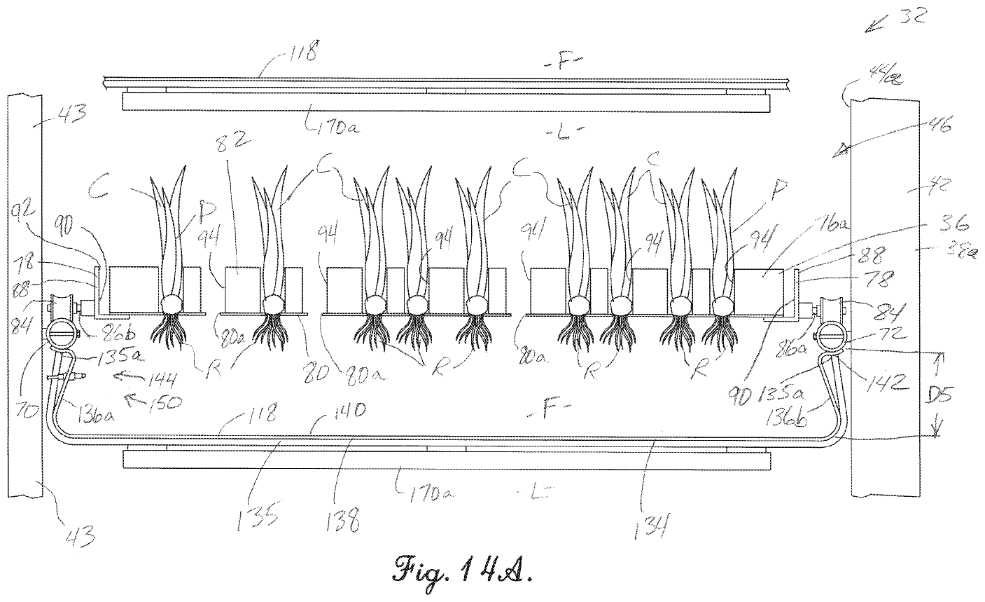

The silos 38 are formed by respective silo walls 42 and define corresponding silo growth chambers 44a,b (see FIG. 3). Each silo wall 42 also defines, in part, a respective spiral growing space 46 that extends within the chamber 44 to receive crops (see FIGS. 3 and 14). Again, although the depicted silos 38 receive plants P and mushrooms M, it is entirely within the scope of the present invention where the silos 38 receive other agricultural crops. As used herein, the term “silo growth chamber” refers to a chamber that is configured to receive various agricultural crops (such as plants, fungi, and/or animals) for growth and/or incubation.

The silos 38 also preferably includes a plurality of vertical support columns 43 arranged about the central access shaft T. Each column 43 preferably comprises a structural beam fixed within the growing space 46. The steel beam preferably includes a structural beam having an I-beam profile. However, the columns could be alternatively constructed, consistent with the scope of the present invention.

The depicted silo growth chambers 44 are each vertically elongated. In particular, each chamber 44 has a generally cylindrical shape that extends vertically and has a circular cross-sectional profile. It will be understood that the silo growth chambers could be variously shaped without departing from the scope of the present invention. For example, one or more silo growth chambers could be shaped to have a profile that is generally square or rectangular.

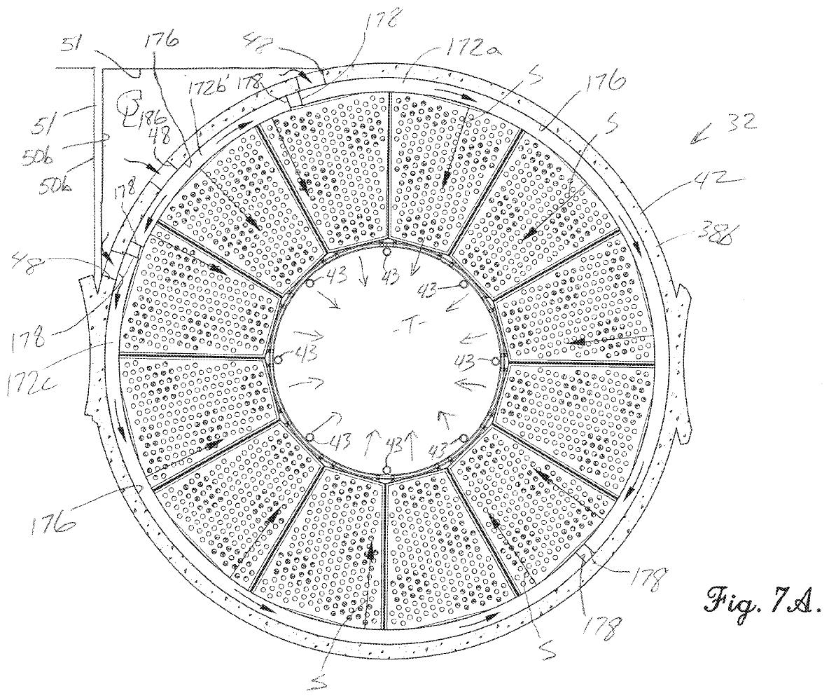

The silo walls 42 also preferably define a group of air inlet openings 48 positioned adjacent to one another (see FIGS. 7 and 7A). The air inlet openings 48 are associated with a respective spiral segment of the growing assembly 32 and are configured to transmit air into the spiral growing space 46. It is also within the scope of the present invention where one or more air inlet openings are configured to, additionally or alternatively, transmit air out of the spiral growing space. The spiral segments in the silo 38 preferably are associated with respective groups of air inlet openings 48. The air inlet openings 48 are preferably configured so that multiple silo growth chambers 44 are in fluid communication with one another (as used herein, the terms “fluid communication” and “fluidly communicate” generally indicate that air is permitted to flow between respective areas).

It will also be appreciated that the silos 38 could have variously configured air inlet openings within the scope of the present invention (e.g., to transmit a desired air flow within the silo). Furthermore, the circumferential range of air ducts need not fully encircle a silo. Air ducts are preferably located in areas with LED lighting in an “on” state.

The silo building 34 is also preferably configured to include a series of interstitial vertical bins 50 (see FIG. 7) located between the silos 38 and defined by respective silo walls 42. As will be discussed, at least one of the interstitial bins 50 is configured to transmit air flow to multiple silos 38 via the groups of air inlet openings 48. In the depicted embodiment, the interstitial bin 50 preferably has one or more internal dividing walls 51 so that the bin 50 has multiple discrete bin chambers 50a,b (see FIGS. 7 and 7a). The bin chambers 50a,b preferably receive cooling air but do not communicate with one another. The bin chamber 50a communicates with two of the silos 38, and the bin chambers 50b communicate with corresponding ones of the silos 38. As will be explained, the bin chambers 50b are supplied with cooling air by respective fan units.

However, it is also within the scope of the present invention where one or more interstitial bins are alternatively configured (e.g., to supply cooling air to silos 38). For instance, the bin could have a larger or fewer number of bin chambers. In alternative embodiments, the bin could have four (4) bin chambers, with each of the bin chambers communicating with only a respective one of the silos. In other alternative embodiments, the bin could have a single bin chamber that provides cooling air to multiple silos.

In various embodiments within the scope of the present invention, the silo growing system is configured to include one or more vertically oriented housings or towers that can range in size. The terms “housing,” “tower,” or “silo” are used interchangeably herein to refer to the depicted silos and denote the vertically oriented, vertically elongated nature of the housing structure, which will typically have a height that is greater than its width. However, for certain aspects of the present invention, the silos (and silo growth chambers) could have an alternative ratio of height and width dimensions (e.g., where the height dimension is less than a width dimension).

In alternative embodiments, one or more silos can be either freestanding or form part of a larger structure or grouping of a plurality of housing structures. In some embodiments, each silo is a cylindrically shaped, vertically elongated structure. Some embodiments of the system will utilize an existing grain silo/elevator or tower as an approach to reusing otherwise vacant structures that are available throughout much of the world. The silo may be made from a variety of materials, such as concrete, steel, and combinations thereof. The most efficient materials are selected to have both high strength and high thermal mass, such as concrete, steel, and the like. The thickness of the silo walls also contributes to the efficiency of the system.

Exemplary thicknesses of the silo walls range from about seven inches (7″) to about ten inches (10″). Silo height may range from about ten feet (10′) up to structural limitations of the selected materials. Preferably, the depicted silos 38 each have a height of at least about one hundred feet (100′), more preferably at least about one hundred twenty feet (120′), and even more preferably at least about two hundred feet (200′). A plurality of individual silo structures can be variously grouped together without departing from the scope of the present invention.

A plurality of individual housing structures are preferably grouped together to increase efficiency and production of hydroponics, aquaculture, and/or aquaponics systems. When grouped together the thermodynamic principals applied to this design are maximized through mechanical and passive heat exchange systems when coupled with environmental control systems of the hydroponics, aquaculture, and/or aquaponics environment.

The adaptive reuse of existing facilities and structures previously used for industrial scale operations, including vertical industrial agricultural structures such as concrete grain elevators/silos and bins, permits direct root application systems to be implemented on an industrial scale in a more energy efficient manner. Additionally, the design and strength of the existing facilities naturally minimizes human labor input and risk. The strength and control design of the facilities allows for the creation of a positive pressure laboratory type environment that provides filtered air flow resulting in minimized impact from pests and plant illness. For example, in some embodiments the air pressure within a silo complex may be maintained at a pressure greater than atmospheric in an effort to reduce external air from entering the silo complex, thereby reducing or even eliminating contaminants from entering the growing operations. The silo walls also preferably provide an impermeable barrier to pest entry.

Although existing structures such as grain elevators/silos and bins are suited for use as the location of the systems described herein, the systems may also be installed in new structures that are designed and constructed specifically for the purpose of housing these types of systems. Some projects may comprise a combination of reuse of existing structures and construction of new complementary structures. It is also within the scope of the present invention, where one or more silos comprise a vertical underground silo, whether the silo is newly excavated or adapted from an existing underground silo.

Moreover, the size, scope and unique design of the facilities allow for the utilization of non-traditional energy sources/methods to, among other things, move and/or power water movement and energy conservation within the facilities. For example, windmills or wind turbines, may be placed on the roofs of the structures (with relatively high wind potential at typical silo heights), and solar panels on the roofs or sides of the structures. Waste heat generated by the unique growth methods can be captured and reused within the facilities (or stored in a climate battery using inexpensive coarse stone in a dedicated thermal storage silo). The height, strength and vertical orientation of the facilities also permit energy recovery via the use of low or reduced energy input pumps and/or technology.

Incorporating a combination of existing and specific design-driven water heating and cooling technology systems and devices placed throughout the facility will control temperature in and around the crops and redistribute captured energy. Capture of waste heat allows for reduced energy needs and further reduction of the facility's carbon footprint. Additionally, because mushrooms release CO2 as a byproduct of their metabolism, growing mushrooms in the system may beneficially increase the CO2 levels within the system needed for photosynthesis by the crops grown within the system. Thus, the transmission of CO2 from the mushroom growth areas may enhance vegetable growth in the remainder of the facility and allow for the processing of waste, both thermal and crop waste from other aspects of the operation.

### Spiral Growing Assembly

Turning to FIGS. 2-6 and 13-14A, the spiral growing assembly 32 preferably includes a series of spiral segments 52 arranged end-to-end (see FIGS. 2, 4, and 5). The series of spiral segments preferably progress downwardly from the top end 64a to the bottom end 64b. These spiral segments 52, or spiral layers, extend a full revolution about a silo axis A1 (see FIG. 3) and thereby provide one “level” of the spiral growing assembly 32. Each pair of adjacent spiral segments 52 at least partly overlap one another along a lateral direction. More preferably, the spiral segments 52 substantially overlap one another.

The spiral growing assembly 32 preferably has about sixty (60) spiral segments 52, although the spiral growing assembly 32 could have more segments or fewer segments. The system 30 is also preferably operable so that the crop supports 36 advance about two (2) revolutions per day. However, the crop supports could advance at a rate less than two (2) revolutions per day or at a rate greater than two (2) revolutions per day. The crop supports 36 are intended to travel the entire length of the spiral growing assembly 32 in an elapsed time that preferably ranges from about twelve (12) days to about sixty (60) days. However, the length of the assembly 32 and/or the speed of the crop supports 36 can be adjusted to increase or decrease the elapsed time (e.g., based upon the needs of the particular agricultural crop).

The spiral growing assembly 32 is positioned in the silo growth chamber 44 and extends along an assembly length 54 (see FIG. 1) to at least partly define the spiral growing space 46, which receives the plants P (and/or other agricultural crops) therein.

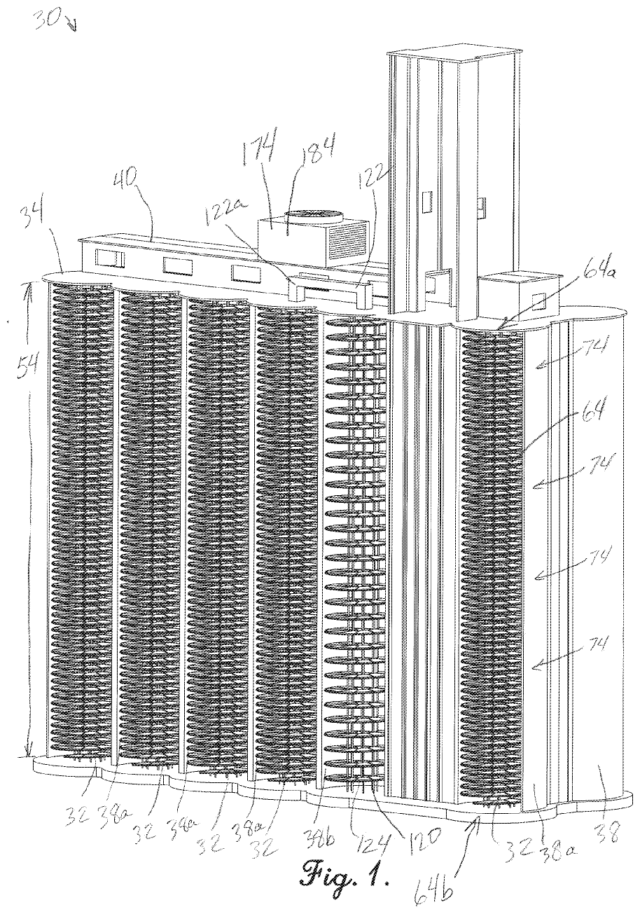

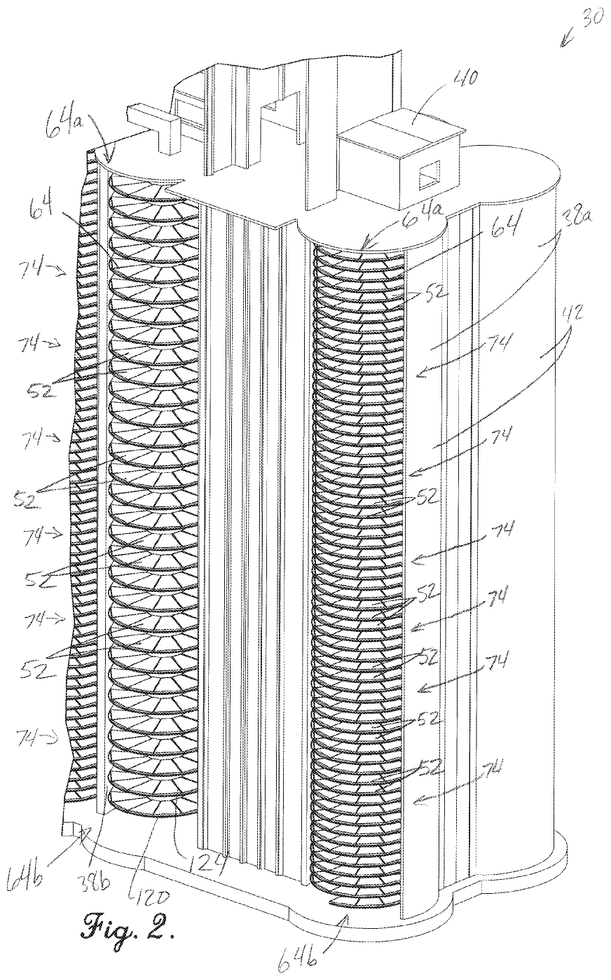

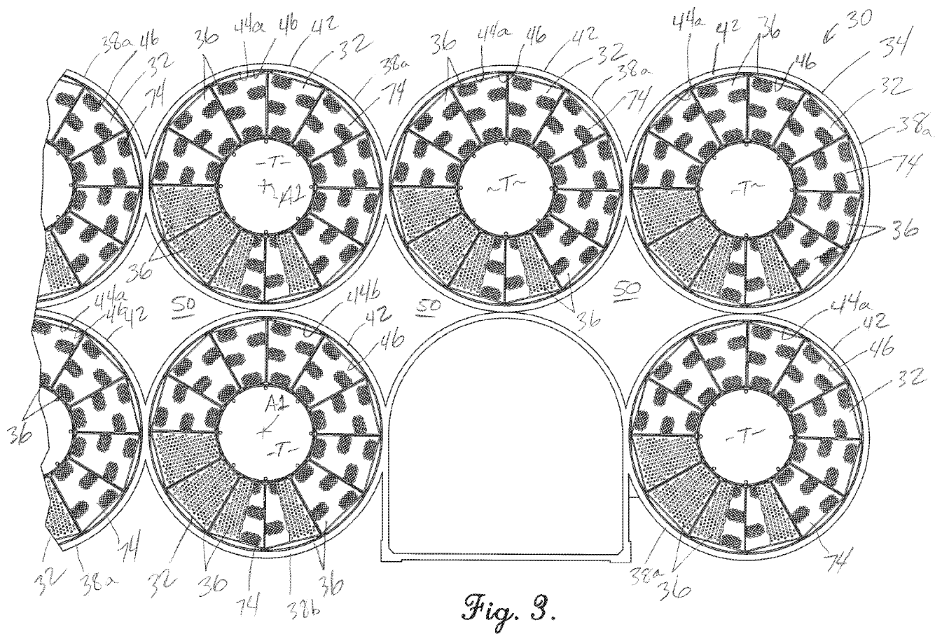

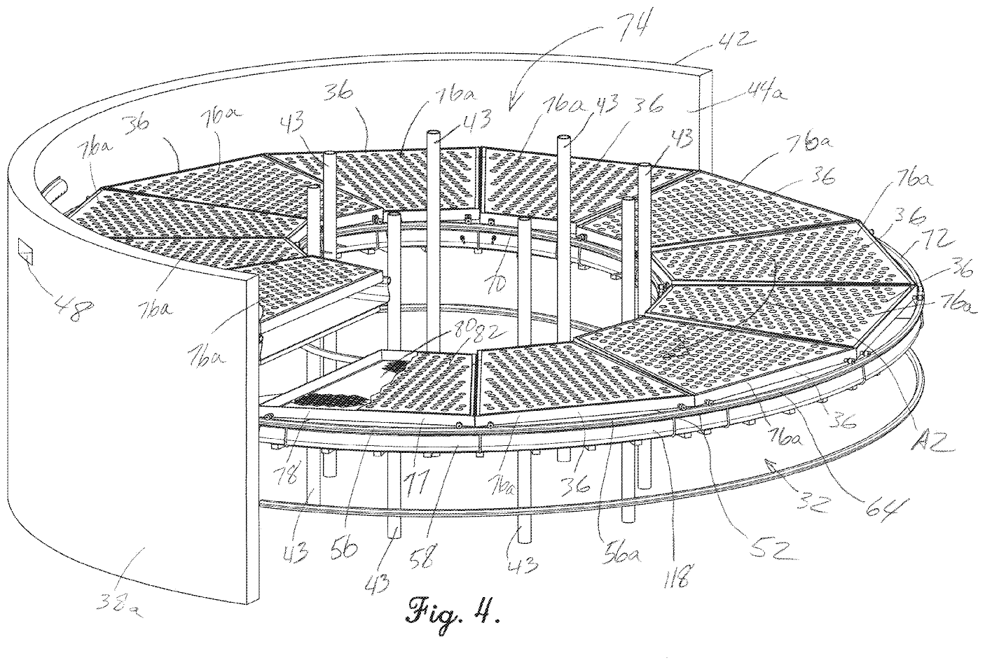

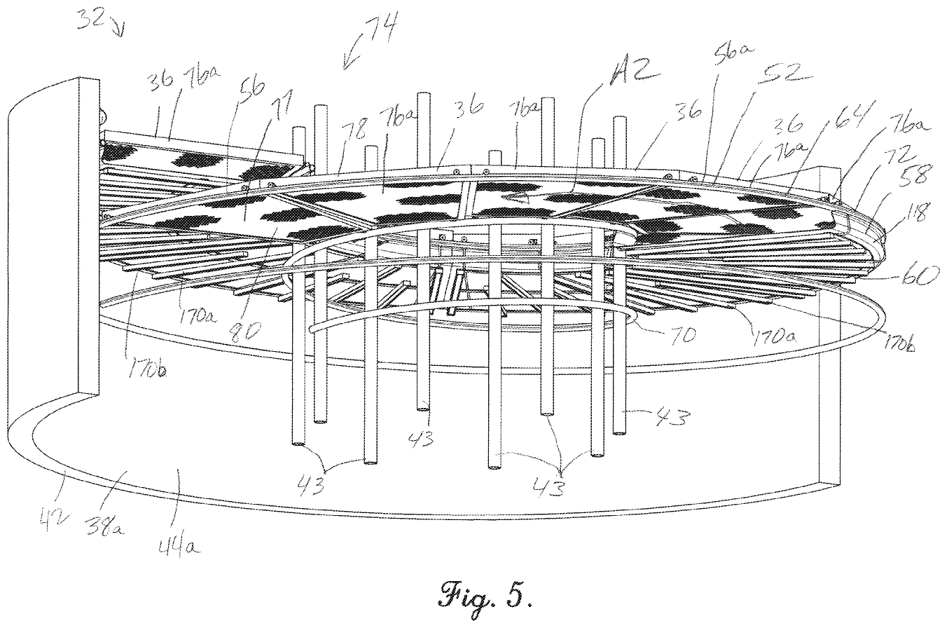

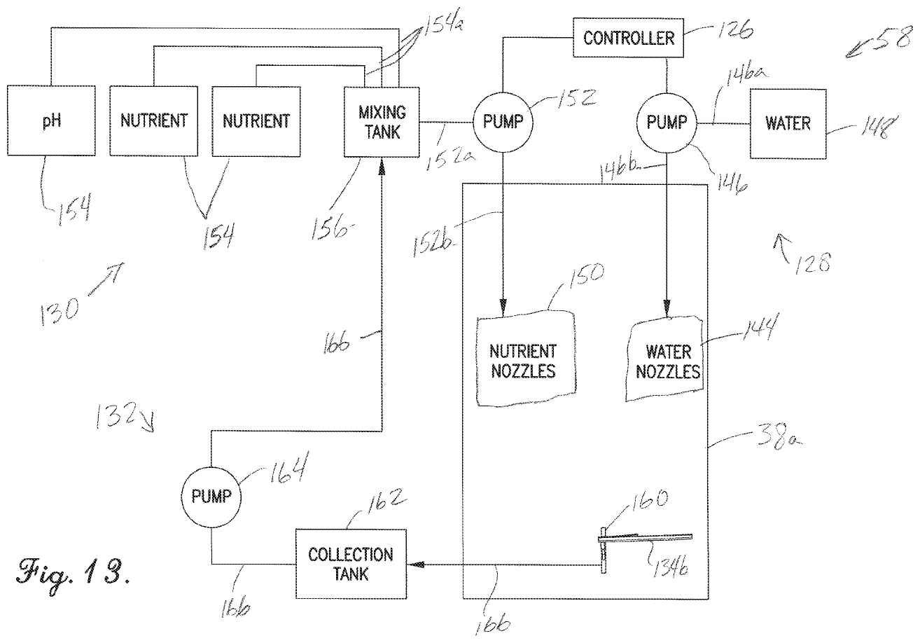

The spiral growing assembly 32 preferably includes a continuous spiral track 56 (see FIGS. 4, 5, and 14), a feeding system 58 (see FIG. 13), a lighting system 60 (see FIG. 5), and an air system 62 (see FIGS. 7, 7A, and 12).

The track 56 extends continuously along the assembly length 54 of the spiral growing assembly 32 and presents a generally downward spiral path 64 that defines a path axis A2 (see FIGS. 4-6). The track 56 is preferably configured to direct the plants P (and/or other agricultural crops) along the spiral path 64.

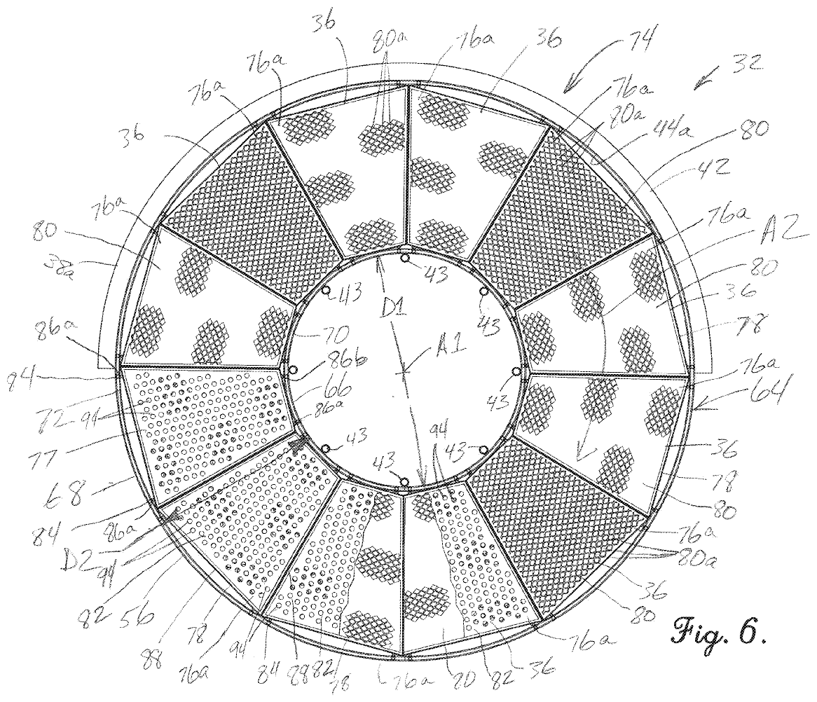

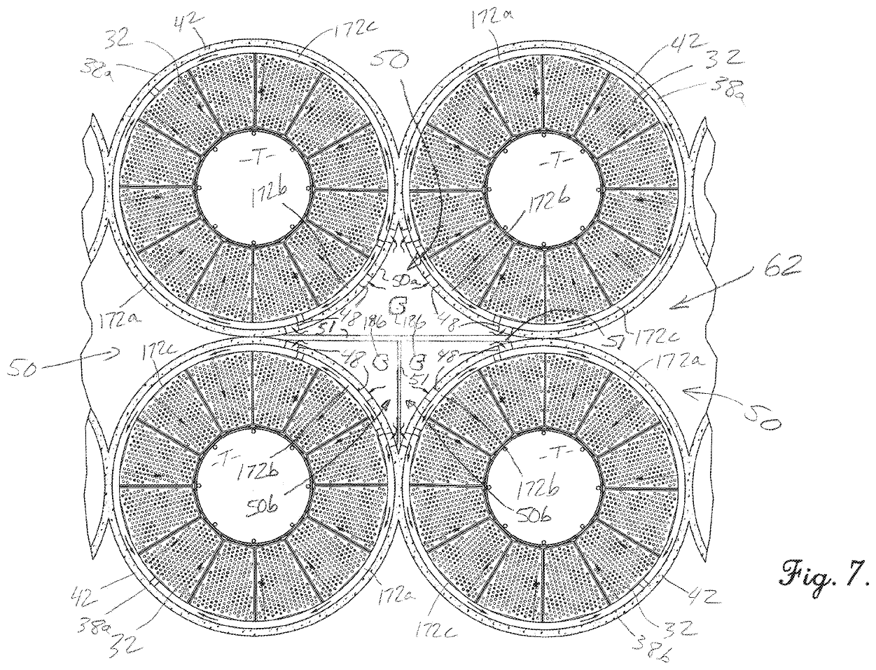

The spiral growing assembly 32 has an inner diameter dimension D1 defined by an inner margin 66 of the spiral growing assembly 32 (see FIG. 6). The inner diameter dimension D1 is preferably substantially constant along the length of the silo axis A1. The inner diameter dimension D1 preferably ranges from about four feet (4′) to about twenty feet (20′) and, more preferably, is about eight feet (8′).

The track 56 also presents a track width dimension D2 measured from the inner margin 66 to an outer margin 68 of the spiral growing assembly 32 (see FIG. 6). The track width dimension D2 preferably corresponds to the width of the spiral path 64.

The spiral shape of the track 56 preferably has a generally circular profile that is designed to follow the shape of the silo wall. It will be understood that the track could have a spiral configuration with various profile shapes without departing from the scope of the present invention. For example, one or more tracks could be shaped to have a profile that is generally square or rectangular. Such an alternative track spiral configuration may be desirable so that the track and silo are complementally shaped (e.g., where the track is installed in a structure having a square or rectangular profile).

The track 56 preferably includes inner and outer rails 70,72 arranged generally parallel to one another and extending along the path axis A2 (see FIGS. 6 and 14). The inner and outer rails 70,72 preferably comprise spiral guide rails to receive the crop supports 36. The inner rail 70 is structurally supported along the central access shaft T by support columns 43. The inner rail 70 is attached to the columns 43 with fasteners and extends from the bottom towards the top of the silo growth chamber 44. The outer rail 72 may be structurally supported along the silo wall 42 surrounding the growth chamber 44 and attached thereto by fasteners.

It will be appreciated that for the same angular displacement around the central shaft T, the inner rail 70 generally descends about the same vertical distance as the outer rail 72, although the inner rail 70 will travel a lesser total distance in its descent than the outer rail 72. Consequently, the pitch (i.e., the angle corresponding to the rise of the rail per unit of horizontal run of the rail) of the inner rail 70 is necessarily greater than the pitch of the outer rail 72. The pitch of the rails 70,72 comprises an angle that preferably ranges from about one degree (1°) to about ten degrees (10°) and, more preferably, ranges from about one degree (1°) to about five degrees (5°). In preferred embodiments, because the pitch of the inner rail 70 preferably is less than about five degrees (5°) and the pitch of the outer rail 72 is a correspondingly smaller amount. For a spiral configuration with a progressively increasing track spacing, the maximum pitch of the inner rail will generally occur adjacent the bottom of the silo where the plant height is generally at the greatest value.

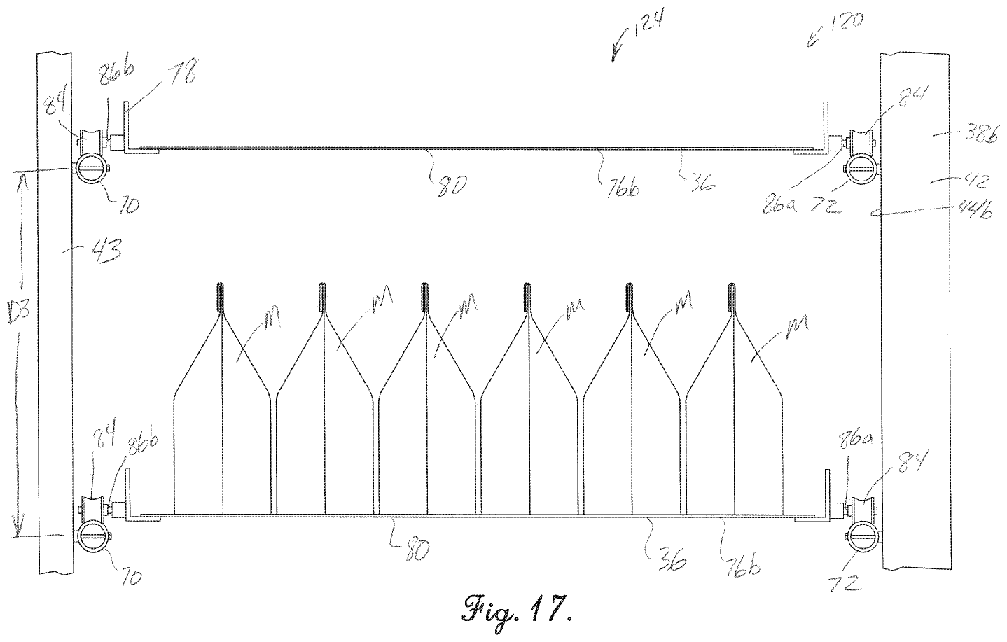

The pitch of the rails 70,72 may be adjusted without departing from the scope of the present invention. For instance, as will be described below, the growing assembly used for mushroom incubation (or incubation of other fungi) has a vertical spacing dimension D3 between adjacent track segments 56a that is preferably substantially constant for each of the inner and outer rails along the spiral path 64 (see FIG. 17).

In various alternative embodiments, it will also be understood that the rail pitch may have a profile that is not constant or progressively increasing along the entire track length in the downward direction. The rails may include one or more rail sections spaced along the assembly length 54 where the pitch changes abruptly to provide a discontinuity in the track (e.g., to facilitate stopping or slowing of one or more carts). For instance, one or more rail sections may be provided along the track where the pitch is approximately zero so that the track has a relatively flat section.

One or more rail sections may also be upwardly pitched to provide relatively more aggressive stopping or slowing of the train. Such upward rail sections may have a slope or pitch that is relatively gradual or relatively sharp, such that the rail section presents a sharp discontinuity in the track.

In a similar manner, it will be understood that one or more rail sections may be spaced along the assembly length 54 to provide a relatively larger pitch (and a relatively larger vertical drop) compared to adjacent rail sections. Such large downward-pitch rail sections may be employed for various reasons (e.g., to facilitate smooth and efficient movement of trains). Large downward-pitch rail sections may have a slope or pitch that is relatively gradual or relatively sharp, such that the rail section provides a discontinuity in the form of one or more discrete stair step rail surfaces. One or more stair step rail surfaces may have relatively sharp corners, such that the rail presents a generally sawtooth rail surface profile. On the other hand, one or more stair step rail surfaces may have rounded or smoothed corners, such that the rail surface profile is undulating with one or more smooth humps. It will be understood that a sawtooth or humped rail surface may be employed for various reasons (e.g., to generally reduce acceleration of carts or to generally adjust the pitch angle of one or more portions of the rail).

The track 56 preferably includes track segments 56a that extend a substantially full revolution about the silo axis A1 and are associated with spiral segments 52 (see FIGS. 4 and 5). A vertical spacing dimension D3 between adjacent track segments 56a (each 360-degree portion) may be provided in various configurations (see FIG. 14). Preferably for the depicted spiral growing assemblies 32, the spacing dimension D3 between the adjacent track segments 56a grows progressively larger from the top 64a of the spiral path 64 to the bottom 64b of the spiral path 64. This progressively increasing spacing is depicted schematically in FIG. 14 (the depiction of the spiral growing assemblies in FIGS. 1 and 2 is schematic, and does not accurately show the progressively increasing spacing between adjacent track segments).

Such a configuration is desirable for crop growth, as younger/smaller seedings/crops will require a shorter vertical space than larger, more mature crops that develop as the crops grow during their movement along the crop support spiral conveyor system from top to bottom.

Most preferably, the spacing dimension D3 preferably increases so that a lighting distance dimension D4 between lights of the lighting system 60 and the tops of the plants P (and/or other agricultural crops) is substantially constant along the spiral path 64 (see FIG. 14). Again, for mushroom incubation, the vertical spacing dimension D3 between adjacent track segments 56a is preferably substantially constant along the spiral path 64 (see FIG. 17).

The minimum vertical spacing between adjacent track segments 56a is limited by the combined height of the bedway, rail height, and the crops being grown. Likewise, the lighting distance dimension D4 can be adjusted as the crops progress through the growth cycle. In addition to the lighting spacing, the lighting spectrum, intensity, and temperature may be variable along the path to optimize light quality for young or mature crops in the life cycle. Different types of crops may also be positioned along the inner edge of a cart or along an outer edge of a cart to best utilize lighting conditions in those areas. Although the progressive spacing dimension D3 is preferably used to maximize the crop density within the silo 38, a generally consistent spacing dimension may also be used by varying the lighting height relative to the top of the crop.

### Movable Crop Supports

Turning to FIGS. 4-6 and 14-14A, each movable crop support 36 is preferably mounted with wheels that rotatably engage the spiral track 56 and move (e.g., via gravity) along the spiral path 64 from the top to the bottom of the vertically elongated silo growth chamber 44.

The movable crop supports 36 are configured to support plants P (and/or other agricultural crops) and be advanced along the track 56 in a controlled manner. The movable crop supports 36 of the illustrated embodiment are preferably arranged end-to-end to cooperatively form at least one train 74 (see FIGS. 3-6). As will be explained, the crop supports 36 are configured to be advanced downwardly along the assembly length 54 under the force of gravity to thereby direct the plants P through the growing space 46 along the spiral path 64. The crop supports 36 also generally separate a feed zone F of the growing space 46 from a lighting zone L of the growing space 46 (see FIG. 14).

The movable crop supports 36 preferably include movable carts 76a,b and a movable control cart 77. As will be described, the depicted train 74 includes pairs of adjacent carts 76,77 that removably contact one another. However, an alternative train could include adjacent pairs of carts 76,77 that are attached.

The carts 76,77 are removably supported on the track 56 and configured to support the plants P (and/or other agricultural crops). The carts 76,77 are configured to be advanced downwardly along the assembly length 54 to thereby direct the plants P through the growing space 46 along the spiral path 64. Preferably, each train 74 includes at least one control cart 77 that controls the speed of advancement of itself and other carts within the train 74. In alternative embodiments, cart advancement is controlled using mechanical restrictions at a series of locations along the track by preferably using, among other things, an electromagnetic device or physical restriction to slow the carts.

The depicted carts 76a,77 of each train 74 are preferably configured for supporting a batch of plants P (and/or other agricultural crops) and includes a frame 78, a mesh bottom 80, a cap 82, and multiple wheels 84 rotatably attached relative to the frame 78 by axles 86a,b (see FIGS. 4 and 14A). The cart 76b is preferably configured for supporting mushroom bags M and includes, among other things, the frame 78, mesh bottom 80, wheels 84, and axles 86a,b.

Within each silo 38a, the frame 78 serves as a structural member to support the cap 82 and plants P as the cart is advanced along the track 56. Similarly, within silo 38b, the frame 78 supports mushroom bags M. The depicted frame 78 includes multiple side members 88 arranged and fixed to one another in a generally trapezoidal shape (see FIGS. 6 and 14A).

However, it is within the scope of the present invention where the frame has an alternative shape, such as an alternative polygonal shape (e.g., a rectangular, triangular, pentagonal, etc.) or another closed figure (e.g., a figure with one or more curved sides).

Each side member 88 has a two-sided angle profile (see FIGS. 14A-16), although the side members 88 could be alternatively formed. For instance, one or more side members 88 could present a generally tubular profile (e.g., a square, rectangular, or circular tubular profile).

The mesh bottom 80 preferably comprises a preformed, expanded metal panel that presents a pattern of diamond-shaped holes 80a that are uniformly arranged and spaced (see FIGS. 6 and 14A). The holes 80a are preferably sized and configured to permit the roots R to extend below the mesh bottom 80 while facilitating crop growth. It is also within the scope of the present invention where the mesh bottom has holes that are alternatively shaped and/or arranged.

The mesh bottom 80 preferably spans the frame 78 and cooperates with the frame 78 to define a cart pocket 90 (see FIG. 14A) to receive the cap 82. The frame 78 also presents an open top 92 (see FIG. 14A) associated with the cart pocket 90.

In alternative embodiments, the carts could be configured without the use of the mesh bottom. For instance, such alternative carts could be devoid of the mesh bottom and have hydroponic plant supports placed into the holes of the cap 82.

For the carts 76a installed in the silo growth chambers 44a, the mesh bottom 80 is configured to support the plants P (and/or other agricultural crops) and permits the roots of the plants P to pass into the feed zone F. For the carts 76b installed in the silo growth chamber 44b, the mesh bottom 80 is configured to support the mushroom bags M.

The frame 78 and mesh bottom 80 preferably comprise a galvanized steel material. However, the frame and mesh bottom could, additionally or alternatively, include another metallic material (e.g., stainless steel or aluminum) and/or a resin material (e.g., a plastic or synthetic resin material).

The cap 82 preferably comprises a unitary structure and presents multiple crop openings 94 (see FIGS. 6 and 14A). The illustrated openings 94 extend through the cap 82 and each have a cylindrical shape with a circular profile. However, one or more openings 94 could be alternatively shaped within the ambit of the present invention. For instance, the opening profile could be oval, polygonal (e.g., square, rectangular, triangular, etc.), tapered, or slotted.

The openings 94 are preferably arranged in a uniform pattern where the openings 94 are spaced apart from one another. It will be appreciated that the openings are preferably spaced to facilitate desired crop growth and/or to maximize the production throughput of crops by the system 30. Consequently, the openings can be arranged in various uniform and/or random patterns (e.g., to provide desired plant spacing).

The openings 94 are configured to receive corresponding ones of the plants P so that plant roots R are generally positioned below the cap 82 and the plant canopy C is generally positioned above the cap 82 (see FIG. 14A).

The cap 82 and the rest of each cart is preferably configured to prevent light from passing through the cart into the feed zone F. Although some of the openings 94 are schematically depicted (particularly in FIGS. 14 and 14A) as being unused and open, it is preferable that any unused openings are generally covered with an opaque material layer (not shown) to prevent light from passing through the cart. Furthermore, to the extent that any gap exists between a plant and the respective opening that receives the plant, it is also preferable that any such gap is generally covered with an opaque material layer to prevent light from passing through the cart. In various embodiments, one or more opaque material layers could be positioned relative to the cap to prevent light transmission through the cart.

The cap 82 preferably comprises an extruded polystyrene (XPS) foam material. The depicted cap 82 is also preferably opaque and spans the spiral path to restrict light from passing through the movable cart 76 and into the feed zone F.

One or more caps could also be alternatively constructed within the scope of the present invention. In alternative embodiments, the cap could comprise a relatively thin layer of synthetic resin material and define a series of slotted openings that provide the crop openings. For instance, each crop opening could be formed by cutting multiple intersecting slots to form a ring of angled tabs that meet at the intersection and form a generally star-shaped opening.

The rear and front-left axles 86a are preferably rigidly mounted to the frame 78 adjacent to respective corners of the frame 78 to rotatably support corresponding wheels 84. The front-right axle 86b is preferably shiftably attached relative to the frame 78 to rotatably and shiftably support another wheel 84 (see FIGS. 6 and 14A). The axle 86b is preferably vertically shiftable so that all wheels 84 of the cart 76,77 ride smoothly on the rails 70,72 and cooperatively support the cart 76,77 on the track 56.

The axle 86b is slidably attached to the frame 78 so as to be vertically movable relative to the frame 78 between an upper position (not shown) and a lower position (see FIG. 14A). The axle 86b is also preferably urged into the lower position by a spring (not shown). In this manner, the respective wheel 84 is urged into rolling engagement with the inner rail 70 while the cart 76 is supported on the track 56. As noted above, the inner and outer rails 70,72 have respective pitches that are different from each other, due to the helical shape of the track 56. The relative difference in pitch between the inner and outer rails may also differ depending on the particular location along the length of the track (e.g., due to a progressively increasing track spacing or another change in track spacing). Preferably, the shiftable axle arrangement enables the cart 76 to be advanced along the track 56 so that all wheels 84 smoothly and continuously engage the track 56.

The depicted wheels 84 are operably engaged with corresponding rails 70,72 and are configured to roll along the rails 70,72 as the movable cart 76,77 is advanced downwardly along the spiral path 64.

Again, the carts 76,77 are preferably arranged in series with one another to form at least one train 74 to hold an agricultural crop (e.g., a batch of plants P). In the depicted embodiment, each pair of adjacent carts 76,77 removably contact one another. Preferably, adjacent pairs of carts 76,77 of the train 74 generally remain in contact with one another (or are closely adjacent one another) as the train 74 advances downwardly along the spiral path 64.

However, in alternative embodiments, the adjacent pairs of carts 76,77 could be attached to one another. For instance, adjacent carts 76,77 could be removably attached to one another with various types of connectors, such as threaded fasteners (e.g., bolts, screws, nuts, etc.), rope, wire, magnets, etc. The connectors preferably comprise connectors that permit relative shifting between the adjacent carts. To this end, the connectors may involve the use of complemental connectors that cooperatively form a shiftable joint between the adjacent carts (e.g., a pivotal joint and/or a sliding joint).

Each train 74 includes a respective series of carts 76,77 that provide a desired train length. The train 74 depicted in FIGS. 4-6 includes twelve (12) carts 76,77 arranged in series, and the train 74 extends substantially one full revolution along the track 56.

However, the trains 74 can be provided with various train lengths without departing from the scope of the present invention. For instance, one or more trains could be longer than one revolution in length or less than one revolution in length. It will be appreciated that the train lengths may be set or adjusted in connection with a desired growth cycle, the particular type of crop being grown, to optimize production throughput, and/or for other suitable operational purposes. Each train 74 is preferably independently controllable of the other trains 74, as will be discussed.

Preferably, the train 74 is configured to be advanced downwardly by the force of gravity. At the same time, the train 74 is operable to control its advancement to facilitate crop growth and achieve desired growth when the train 74 reaches the bottom of the spiral growing assembly 32.

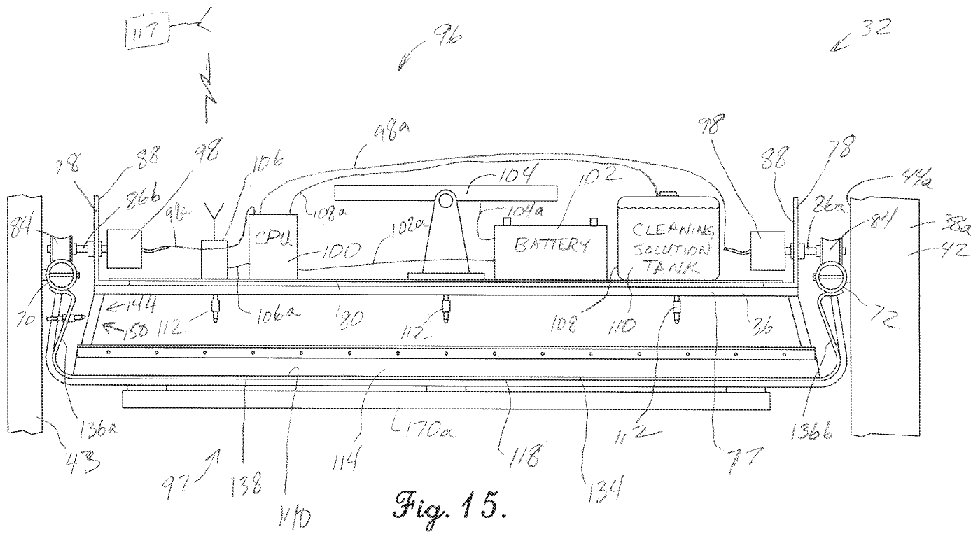

Turning to FIG. 15, the control cart 77 is positioned in front of the other carts 76 within the train 74 and preferably includes a braking mechanism 96 and a bedway cleaning device 97. The braking mechanism 96 is operable to control the control cart 77 and facilitate advancement of the train. The braking mechanism 96 comprises a pair of conventional friction brakes 98 associated with corresponding wheels 84 of the cart 77. The brakes 98 can be selectively fully engaged to prevent rotation of the wheels 84 and selectively disengaged to permit free wheel rotation. Additionally, the brakes 98 can also be progressively engaged between disengagement and full engagement to permit limited wheel rotation.

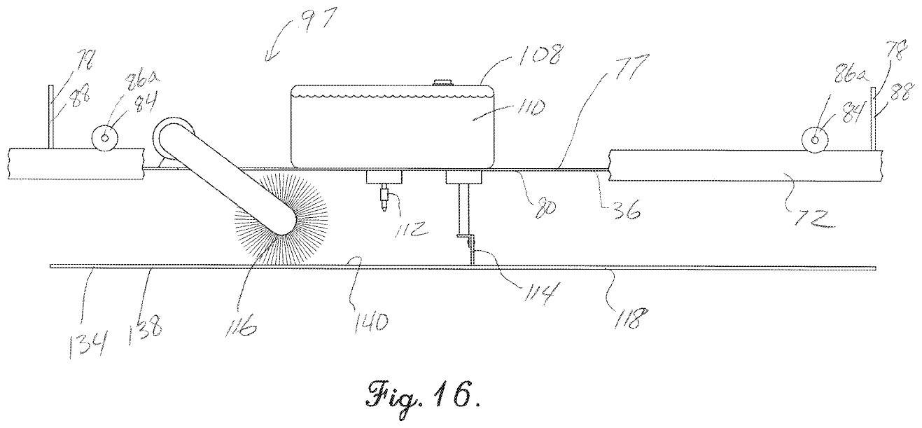

The depicted control cart 77 also includes an on-board CPU 100, battery 102, solar panel 104, and transceiver 106 to facilitate operation of the braking mechanism 96 and the cleaning device 97. The cleaning device 97 preferably includes a tank 108 with cleaning solution 110, nozzles 112 to dispense the cleaning solution 110, a squeegee 114, and a powered rotating brush 116 (see FIGS. 15 and 16).

The brakes 98 are operably coupled to the CPU 100 to control and facilitate advancement of the cart 77. The CPU 100 is powered by the battery 102 via line 102a. The CPU 100 selectively operates the brakes 98 by providing power to release the brakes 98 when movement is desired (i.e., the brakes are preferably normally engaged). When engaged, the brakes 98 restrict rotation of the front wheels 84 and thereby restrict the cart 77 from rolling along the track 56. When disengaged by the CPU 100, the brakes 98 permit free rotation of the front wheels 84 and thereby permit the cart 77 to roll along the track 56.

It will be understood that the CPU 100 is configured to control the brakes 98 via lines 98a and operate the brakes 98 so that the cart 77 can descend at any of a range of speeds and can also be selectively stopped on the track 56. The brakes 98 may be configured to be progressively engaged by the CPU 100 to provide a corresponding reduction in cart speed. Similarly, the brakes 98 may be configured to be progressively disengaged by the CPU 100 to provide a corresponding increase in cart speed.

For instance, the cart 77, and the rest of the corresponding train 74, preferably travels through about two (2) revolutions (or levels) of the track 56 per day. However, it is within the ambit of the present invention where the cart 77 is advanced at a slower or faster speed along the track 56. For instance, it will be appreciated that the speed of the control cart 77 may be adjusted to facilitate optimum growth of different types of crops.

The CPU 100 is operably coupled to the transceiver 106 and thereby preferably communicates wirelessly with a central control station 117. The CPU is configured to receive various operation commands from the station 117 and to transmit various operation data to the station 117. It will also be appreciated that various operation commands and operation data may be stored in memory (not shown) provided as part of the cleaning device 97 and stored on board the control cart 77. It is within the ambit of the present invention where the system 30 utilizes alternative control and/or communication equipment to operate various features of the cart 77.

Also, the control carts 77 are preferably independently controllable of each other. In this manner, each train 74 is preferably independently controllable of the other trains 74 to permit desired advancement of plants P (and/or other agricultural crops) along the assembly length 54.

The principles of the present invention are equally applicable where control carts 77 of multiple trains 74 communicate with one another and/or communicate with the station 117 to coordinate operation with one another. For instance, the carts 77 could communicate with each other to coordinate their advancement along the same spiral path 64 (e.g., so that the carts 77 advance at the same time and/or the same speed).

Each train 74 preferably includes a single control cart 77 positioned in front of one or more carts 76 to provide a lead cart that controls train advancement. However, it is within the scope of the present invention where the train 74 includes multiple control carts 77 arranged in series as lead carts. It will be appreciated that multiple control carts could be configured to alternatively or cooperatively control advancement of the train 74.

Furthermore, one or more control carts could be alternatively positioned along the train to control train advancement. For instance, a control cart could be positioned between a pair of carts 76 or at the back end of the train. In such alternative embodiments, it will be understood that all carts in the train may need to be attached to one another (or otherwise connected) to restrict one or more carts from being separated from one another.

For certain aspects of the present invention, one or more trains could be devoid of a control cart. For instance, the train (or individual carts) could be coupled to an external motorized drive system that is not part of the train.

In general, the illustrated carts 76 preferably provide no braking control (or driving control) over train advancement. However, for certain aspects of the present invention, one or more carts 76 could be provided with a braking mechanism and/or drive mechanism.

Again, the braking mechanism 96 is configured to facilitate advancement of the train 74 along the spiral path 64. Although the control cart 77 is preferably advanced downwardly along the track 56 by gravity and by operating the braking mechanism 96, it is within the scope of the present invention where the control cart 77 is selectively powered along the track 56 to facilitate advancement of the train. For instance, the control cart could include a powered motor (e.g., an electric motor, hydraulic motor, etc.) that operably powers at least one of the wheels 84 to drive the control cart and thereby advance the train along the track 56.

Again, for some aspects of the present invention, the train could be powered by an external motorized drive system that is not part of the train itself. For instance, the depicted silo growing system could have a motorized conveyor system operably supported along the track to drive one or more carts of the train. In various embodiments, a continuous conveyor system could include one or more wheels and/or endless drive elements (such as a chain, belt, rope, etc.) to engage and drive one or more movable carts. In such an alternative conveyor system, the carts could each comprise a unitary tray that is removably engaged by the conveyor system.

The solar panel 104 is electrically coupled to the battery 102 via line 104a and is configured to charge the battery 102 when exposed to light. In the depicted embodiment, the solar panel 104 is preferably exposed to the lighting system 60, which causes the solar panel 104 to charge the battery 102. Thus, as the cart 77 is advanced along the track 56, the LED lights associated with the lighting system 60 charge the solar panel 104, which in turn charges the battery 102.

It is also within the scope of the present invention where the control cart includes an alternatively configured solar panel or is devoid of the solar panel. For instance, the control cart could be operable to be powered only by the battery 102. In other alternative embodiments, the control cart could receive power from another power source. For example, the system 30 could include an electrical power line that extends along the length of the track 56 to power the control cart. In such embodiments, it will be appreciated that the control cart could draw power from the power line using various mechanisms. For instance, the cart could draw electrical power through direct contact with the power line (e.g., with an electrical brush configuration), through induction, or by some other means.

Turning to FIGS. 15 and 16, the control cart 77 is also preferably configured to clean a collection bedway 118 of the feeding system 58. The squeegee 114 is preferably mounted to the frame 78 and configured to engage the bedway 118 as the control cart 77 is advanced. The squeegee may be retractable to clear hazards that may be mounted to the bottom of the collection bedway.

The depicted tank 108 holds a supply of cleaning solution 110 and includes a pump (not shown) that is powered by the battery 102 and fluidly discharges solution via the nozzles 112. The nozzles 112 are operable to dispense the cleaning solution 110 onto the bedway 118 as the control cart 77 is advanced. The cleaning solution 110 preferably comprises a hydrogen peroxide solution but could include, alternatively or additionally, other cleaning solutions suitable for cleaning algae growth, excess fluid, and/or other foreign matter from the bedway 118.

The rotating brush 116 is configured to engage and clean the bedway as the control cart 77 is advanced. The rotating brush 116 is operably coupled to an electric motor (not shown), which is powered by the battery 102. The brush 116 can be selectively powered to rotate and engage the bedway 118. The brush 116 is particularly useful for removing algae growth or other solid foreign matter from the surface of the bedway 118.

The trains 74 in the silos 38b preferably each have a fungi control cart (not shown) similar to control cart 77 to control the advancement of mushrooms (or other fungi). However, the fungi control cart preferably does not include a cleaning device similar to the cleaning device 97 of the control cart 77. The fungi control cart also preferably does not include a solar panel. The fungi control cart otherwise includes features similar to the control cart 77.

The principles of the present invention are equally applicable where one or more of the carts 76,77 are alternatively configured to advance crops along the assembly length. For instance, the carts 76,77 could include an alternative wheel arrangement for being movably supported on the track.

For certain aspects of the present invention, one or more of the movable crop supports could be devoid of wheels. For example, each movable crop support could comprise a single, unitary support structure. In alternative embodiments, the movable crop supports could be slidably engaged with the track in various configurations. For instance, a series of movable crop supports could be mounted on a powered conveyor drive to cooperatively provide a conveyor system. It will also be understood that one or more trains of carts could assume other alternative configurations without departing from the scope of the present invention.

In operation, each train 74 is generally loaded onto the track 56 adjacent a top end 64a of the spiral path 64. In the depicted embodiment, the gallery 40 provides a loading area (not shown) for an operator to load the carts of the train 74 in series. Initially, the control cart 77 is loaded onto the track 56. The control cart 77 is then advanced slowly along the track 56 to allow multiple carts 76 to be loaded successively onto the track 56 behind the control cart 77.

It will be appreciated that the carts 76,77 can be loaded onto the track 56 with plants P, mushrooms M, or other agricultural crops being pre-positioned thereon. If the carts 76,77 are pre-positioned on the track 56 in an empty condition, the gallery 40 is preferably configured to allow the operator to place plants P or mushrooms M (and/or other agricultural crops) onto the pre-positioned cart.

With one train 74 being loaded and advanced along the track 56, one or more additional trains 74 can then be loaded onto the track 56 adjacent the top end 64a.

As discussed above, the control cart 77 generally controls advancement of the corresponding train 74 downwardly along the spiral path 64 from the top end 64a to a bottom end 64b. With multiple trains 74 loaded onto the track 56, the corresponding control carts 77 can each be advanced to move the trains 74 downwardly. For instance, the control carts 77 of multiple trains 74 can be simultaneously advanced so that the trains 74 are advanced at the same time (e.g., to avoid the trains 74 from colliding with one another). It will also be appreciated that the control carts 77 can be advanced at different times while advancing the trains 74, although the trains 74 are preferably prevented from colliding with one another. Additionally, the control carts 77 are also preferably advanced at substantially the same speed, although the carts 77 can be advanced at different speeds within the scope of the present invention.

As the train 74 approaches the bottom end 64a, an unloading or harvesting area (not shown) is provided for the operator to unload the carts off the track 56. The unloading or harvesting area may also be referred to as an unloading or harvesting zone. It will be understood that the terms “unloading” and “harvesting” are generally interchangeable, as used herein, although such terms may also be used in connection with fungi and plants, respectively.

The carts 76,77 of the train 74 are preferably removed starting with the control cart 77. The train is then allowed to index single carts to the harvesting or unloading zone one at a time, under operator or automated control, or a combination thereof. With the lead cart positioned in the harvesting zone, the crop portion on the lead cart is harvested from the lead cart. The lead cart is removed from the track after harvest, allowing the next cart in line to move into the harvesting zone for harvesting the crop portion thereon. In this manner, the crop portions are removed from each cart in the train, and each cart is removed from the track after harvest.

It will be appreciated that the carts 76,77 can be unloaded from the track 56 with plants P, mushrooms M, or other agricultural crops remaining thereon. However, the unloading area is preferably configured to allow the operator to remove the crops from the cart prior to unloading the cart from the track 56.

### Mushroom Production Assembly and Return Air System

Turning to FIGS. 2 and 17, the silo growing system 30 also preferably includes a mushroom production assembly 120 and a return air system 122 associated with silo growth chamber 44b. The mushroom production assembly 120 is preferably positioned in the silo growth chamber 44b and is configured to incubate mushrooms M therein. However, it is equally within the ambit of the present invention where fungi other than mushrooms M are incubated in the assembly 120, such that the assembly provides a fungi production assembly.

The mushroom production assembly 120 preferably includes a spiral growing assembly 124 and multiple crop supports 36 located in silo growth chamber 44b. The spiral growing assembly 124 preferably includes segments 52, a spiral track 56, and an air system 62 similar to spiral growing assembly 32. However, the spiral growing assembly 124 preferably does not include a feeding system 58 or a lighting system 60. As discussed, the crop supports 36 for the mushrooms M preferably comprise carts 76b. The air system for the mushrooms (or other fungi) may have air distribution to homogenize the air temperature and bleed heat from the mushroom bags (or other fungi production). This air will preferably be fed through the walls in a similar manner to the plants P and will preferably use HEPA filters to ensure the mushroom incubation air is of a reduced contaminant quality.

As noted above, the track 56 of the spiral growing assembly 124 has a different configuration than the track 56 of the spiral growing assembly 32. In particular, the vertical spacing dimension D3 between adjacent track segments 56a is preferably substantially constant along the spiral path 64 (see FIG. 17). Furthermore, the spiral growing assembly 124 has about thirty (30) spiral segments 52, although the spiral growing assembly 32 could have more segments or fewer segments.

The illustrated silo growing system 30 includes a single mushroom spiral growing assembly 124 and silo growth chamber 44b for mushroom incubation. However, it is also within the ambit of the present invention where the system 30 includes multiple silo growth chambers 44b for mushrooms M (or other fungi), along with corresponding growing assemblies housed in the silo growth chambers 44b to support the mushrooms M (or other fungi) therein. The system 30 may include an alternative configuration and/or number of mushroom silos for various purposes (e.g., to provide carbon dioxide and heat for optimizing crop growth in the other silos).

The return air system 122 is preferably configured to facilitate fluid communication between the silo growth chamber 44b used to incubate the mushrooms M and silo growth chambers 44a configured to grow plants P (and/or other agricultural crops) (see FIG. 1). In particular, the return air system 122 includes a duct 122a and a powered fan (not shown) and is configured to transmit air from the silo growth chamber 44b for the mushrooms M to a silo growth chamber 44a for plants P (and/or other agricultural crops) (see FIG. 1). It will be appreciated that the return air system 122 enables heat and carbon dioxide produced by the mushrooms M to be transmitted from the silo growth chamber 44b to plants P in the silo growth chamber 44a. When air is supplied from the mushrooms to plants P, the makeup air will be supplied to the mushrooms M by a HEPA filtered air plenum. This supply plenum can also act as temperature conditioning if the mushroom zone requires heat to be removed by venting out of the mushroom silo.

Although not depicted, the return air system 122 could also be configured to transmit air from the silo growth chamber 44b to multiple adjacent silo growth chambers 44a. In various embodiments, it will be understood that the return air system 122 may include additional ducts and/or fans to suitably transmit air to the silo growth chambers 44a. For certain aspects of the present invention the system 30 could also be devoid of the mushroom production assembly.

### Feeding System

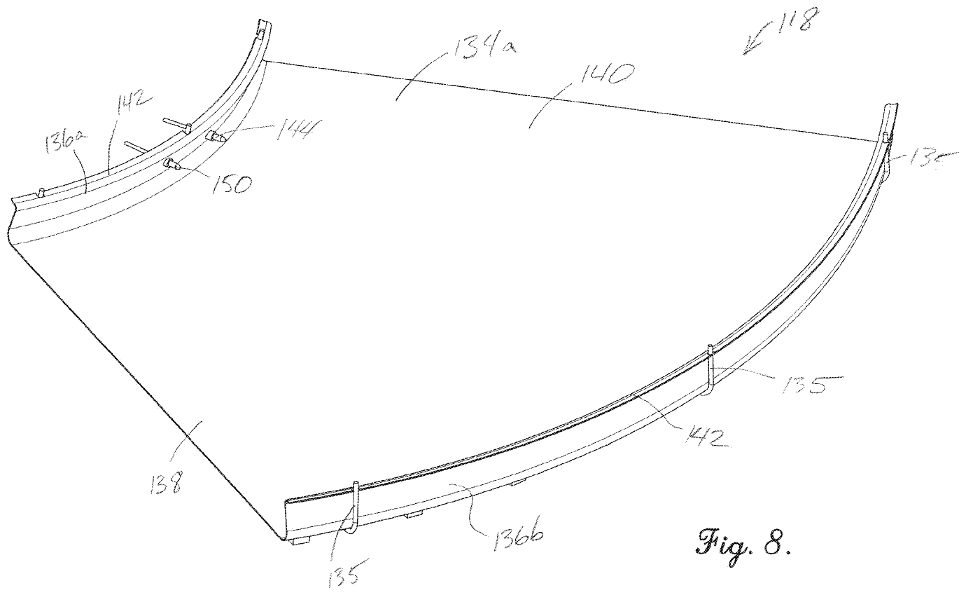

Turning to FIGS. 8-10, 13, and 14-14A, the depicted feeding system 58 is configured to provide water and/or nutrients to the plants P (and/or other agricultural crops) as the crops are advanced along the spiral path 64 (see FIG. 13). The feeding system 58 extends along the track 56 to direct a supply of water and/or nutrients into the growing space 46 along the spiral path 64 and provide the supply to the plants P (and/or other agricultural crops).

The feeding system 58 preferably cooperates with the track 56 to define the feed zone F therebetween. The feed zone F is configured to at least partly receive the plants P and permit application of the supply of water and/or nutrients to plants P inside the feed zone F. The feeding system 58 is also preferably positioned below the track 56 at locations along the assembly length 54.

The feeding system 58 preferably includes the spiral bedway 118, a controller/CPU 126, a water system 128, a nutrient system 130, and a liquid return system 132 (see FIGS. 13 and 14A).

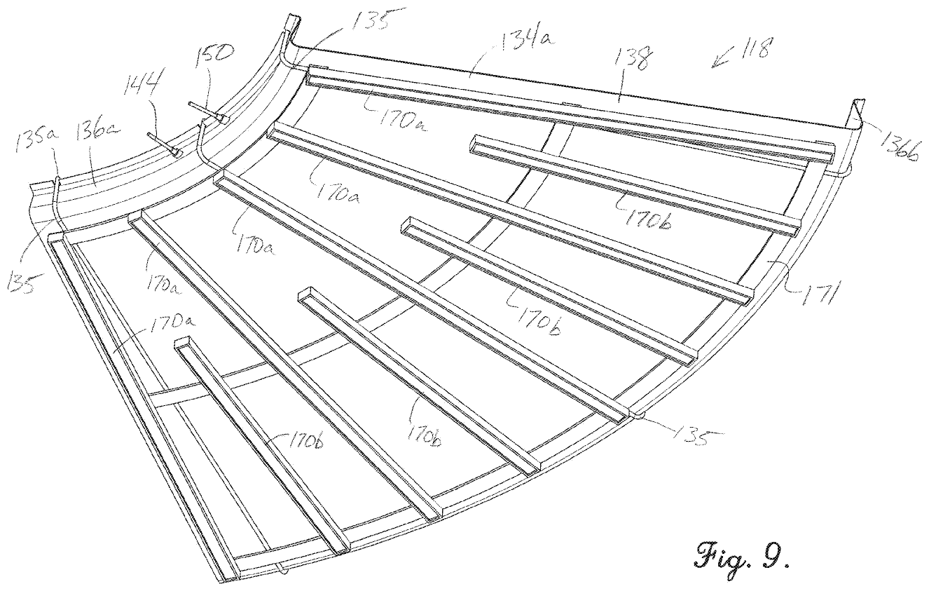

Extending along the spiral path 64 between the top 64a and bottom 64b, the spiral bedway 118 extends radially between the inner rail 70 and the outer rail 72. Preferably, the spiral bedway 118 is cooperatively formed by a series of collection trays 134a,b that are generally arranged end-to-end (see FIGS. 4-5 and 8-10). The bedway 118 also preferably includes a plurality of elongated supports 135 that cooperatively support the trays 134a,b (see FIGS. 9, 14, and 14A).

The supports 135 each comprise an elongated metal rod with opposite rod ends 135a (see FIG. 14A). The rod ends 135a are attached to corresponding rails 70,72 so that the support 135 is securely fixed to the track 56. However, the supports 135 could be variously mounted (or integrated with the bedway 118) without departing from the scope of the present invention.

Each tray 134a,b includes an inner circumferential sidewall 136a, an outer circumferential sidewall 136b, and a bottom wall 138 that extends therebetween and serves as a floor (see FIGS. 8-10 and 14A). The sidewalls 136 and bottom wall 138 cooperative define a channel 140.

The bedway 118 is preferably positioned below the track 56 at locations along the assembly length 54. The sidewalls 136 of the trays 134 are attached to respective ones of the rails 70,72 with fasteners (not shown). The sidewalls 136 each present an upper margin 142 of the tray 134 (see FIG. 14A). The upper margin 142 and bottom wall 138 cooperatively define a channel height dimension D5 (see FIG. 14A). The sidewalls 136 are preferably flexible (i.e., expandable and contractable) so that the channel height dimension D5 may increase and/or decrease so as to accommodate crop root depth. For instance, the flexible construction of the sidewalls 136 preferably permits the channel 140 to expand as the roots R of plants P (and/or other agricultural crops) grow and come into contact with the bottom wall 138.

Any suitable water-proof material can be used to form the trays. Preferably, the tray 134 is dark colored or black (or painted to have a dark color) to reduce irradiance from any light leakage, which is known to inhibit algae growth. An exemplary material used to form the tray 134 comprises an ABS plastic. However, the trays can, additionally or alternatively, include one or more other synthetic resin materials and/or a metallic material within the ambit of the present invention.

In one or more embodiments, the tray 134a,b comprises a plurality of wedge- or trapezoid-shaped segments connected in an overlapped (shingle) configuration to form a downward spiral for the flow, by gravity, of excess water dripping from the misted crop roots R. Accordingly, the tray 134 is positioned underneath the crop supports 36 and has a general U-shaped cross-section between its inner and outer circumferential edges (e.g., like a spiral slide or chute). The interior and/or exterior circumferential edges of each individual tray may be straight or curved as desired.

According to a preferred embodiment, the trays 134a,b are shaped to have a uniform, predetermined downward pitch, which allows the trays to be overlappingly-joined to form the helix spanning the desired height. Again, the pitch can be selected so that the spiral bedway 118 provides a desired number of full or part turns for a given height (vertical distance) change.

Excess water may flow downward along the trays 134a,b. At least one tray 134b is preferably positioned at each level of the bedway 118 to collect excess water associated with that level of the bedway 118. As will be discussed, the tray 134b is attached to a collection gutter associated with the return system 132. It will also be understood that at least some excess water may flow to the bottom of the bedway 118.

The bedway 118 cooperates with the track 56 to define the feed zone F therebetween. The bedway 118 is generally positioned below the track 56 at locations along the assembly length 54. The feed zone F is configured to at least partly receive the plants P (and/or other agricultural crops) and permit application of the supply of water and/or nutrients to the crops inside the feed zone F.

Turning to FIGS. 8-10 and 13, the water system 128 is operable to direct a supply of water into the growing space 46, while the nutrient system 130 is operable to direct a supply of a nutrient solution into the growing space 46.

The water system 128 preferably includes a plurality of dispensing water nozzles 144, a water pump 146, and a water container 148 (see FIG. 13). The water container 148 holds the water supply and communicates with the water nozzles 144 via the water pump 146, pump line 146a, and misting lines 146b (see FIG. 13). The water pump 146 is operated by the controller 126 to selectively draw water from the container 148 and dispense water through the water nozzles 144. The water nozzles 144 are configured to dispense water inside the feed zone F (see FIG. 14A). As mentioned, the bedway 118 is configured to collect any excess part of the supply of water.

The nutrient system 130 preferably includes a plurality of dispensing nutrient nozzles 150, a nutrient pump 152, nutrient solution containers 154, and a mixing tank 156 (see FIG. 13). The nutrient solution containers 154 hold respective supplies of nutrient solutions. Nutrient solutions can be supplied to the mixing tank 156 via fluid lines 154a as needed for mixing in a predetermined ratio. The mixing tank 156 communicates with the nutrient nozzles 150 via the nutrient pump 152, pump line 152a, and misting lines 152b (see FIG. 13).

The depicted nutrient pump 152 is operated by the controller 126 to selectively draw the mixed nutrient supply from the mixing tank 156 and dispense the nutrient supply through the nutrient nozzles 150. The nutrient nozzles 150 are configured to dispense nutrient solution inside the feed zone F (see FIG. 14A). The bedway 118 is configured to collect any excess part of the dispensed nutrient solution.

The dispensing nozzles 144,150 are configured to cooperatively provide a direct root spraying system (e.g., aeroponics, fogponics, etc.) and facilitate direct root application of water and/or nutrients. In this manner, it will be understood that the dispensing nozzles 144,150 may include a sprayer, mister, and/or fogger. In alternative embodiments, fog can be produced outside of the silo growing chamber to include water and/or nutrients and then introduced to the feed zone via ducting and waterproof fans. For instance, the fog could be produced within and/or transported through the interstitial bin 50. It will be appreciated that a combination of spraying, misting, and/or fogging methods may be employed simultaneously.

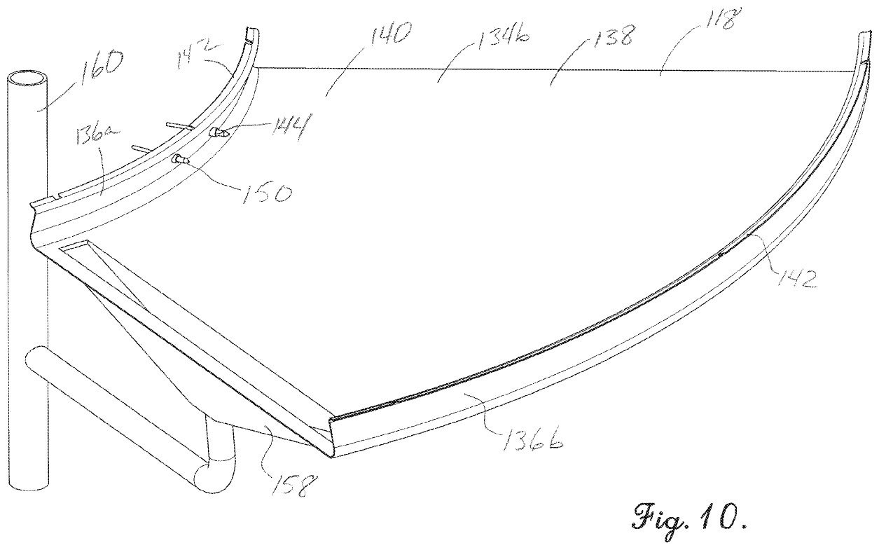

Turning to FIGS. 10 and 13, the return system 132 is configured to collect any excess amount of water and/or nutrient solution from the spiral bedway 118 and convey the excess amount to the mixing tank 156. The return system 132 includes multiple collection gutters 158 associated with the trays 134b, a common downspout 160, a collection tank 162, a return pump 164, and return fluid lines 166 (see FIG. 13).

The collection tank 162 fluidly communicates with the spiral bedway 118 via the gutters 158 and downspout 160 to collect the excess water and/or nutrient solution. The return pump 164 selectively draws excess fluid from the collection tank 162 and discharges the excess fluid into the mixing tank 156.

At least one gutter 158 is preferably positioned at each level of the bedway 118 to collect excess water associated with that level of the bedway 118. However, the return system 132 could have an alternative number and/or arrangement of gutters 158 to collect water. It will also be understood that at least some excess water may flow to the bottom of the bedway 118. In various embodiments, a gutter could be positioned below a location where nutrients are applied to divert run-off water with high nutrient content directly to the nutrient tank. Another gutter can be positioned to direct mist water run-off directly to the water misting system.

It is also within the scope of the present invention where the return system has an alternative configuration of water outlets and/or downspouts provided at various locations along the inner or outer circumferential edge of the bedway (or any intermediate position in between).

At various intervals along the inner and/or outer circumferential sidewalls 136a,b of the tray 134, nozzles 144,150 are provided so that the nutrient spray or mist can be delivered into the space between the tray 134 and the crop supports 36 (and thus the root system of the crops, which extend down into this space underneath the crop supports). Preferably, the nozzles 144,150 are preferably positioned on the inner sidewall 136a of the tray 134, wherein the water and nutrient droplets are sprayed radially outwardly.

Although the depicted nozzle arrangement is preferred, the water system and/or the nutrient system may utilize a multi-nozzle spray wand, a spray plenum, and/or another spraying device.

The direction of spray may be adjusted so that the nozzle (and thus the spray) is directed “uphill,” or “downhill,” or in a direction perpendicular to the central access shaft. Preferably the crop supports 36 and trays 134 are positioned relative to one another to minimize (and preferably avoid) contact between the crop roots R and the trays 134 to permit the roots R to adequately dry between mi stings.

Aeroponics benefits greatly from atomized or small particle sizes of water with dissolved nutrients. NASA determined the optimum particle size was fifty (50) microns. With one hundred feet (100′) of head there is forty-three (43) psi in pressure loss. Currently high pressure aeroponics technology benefits from providing a nozzle supply pressure of at least eighty (80) psi. Therefore, the water and nutrient pumps 146,152 preferably discharge to respective pump lines at a pressure of about one hundred fifty (150) psi to ensure enough line pressure at the highest point of the growth chamber.

Misting lines 146b,152b can run vertically along a vertical cable tray attached relative to support columns 43 or other support structure. The lines can support eight (8) levels but with redundancy. Again, the depicted nozzles are placed on the sidewalls of the bedway and point radially outwardly. The mist is projected outwardly in a cone shape to match the wedge-shape of the crop support structures and ensure even coverage. Although not depicted, the water system 128, nutrient system 130, and return system 132 may each include redundant pumps and/or redundant lines so that the system remains operational even during mechanical failures.

Again, the nozzles 144,150 preferably produce a mist/spray for direct root spraying associated with aeroponics and/or fogponics. Additionally or alternatively, for certain aspects of the present invention, water and/or nutrients could be applied using other direct root application methods, such as nutrient film technique (NFT). In such alternative embodiments using NFT, a supply of water and liquid nutrients can be dispensed to flow continuously along the channel 140, with the roots R being in contact with the flow. It will be appreciated that the flow and water and liquid nutrients can be introduced to the channel 140 using various nozzles or other types of dispensing equipment.

In connection with the use of NFT, the return system 132 may be variously modified to facilitate a suitable flow of water and nutrients along the channel 140. For instance, the depicted gutters 158 may be modified or removed entirely to provide a desired flow of water and nutrients.

In operation, the disclosed feeding system 58 is operable to provide an air/mist environment, utilizing direct root spraying systems. The trains 74 of carts 76 are configured to support and advance plants P (and/or other agricultural crops) along the assembly length 54 while the crops are fed during feeding intervals spaced along the assembly length 54.

In the case of vegetables, as well as other plants (and/or other agricultural crops), the crop supports 36 are configured so that the body, leaves, and/or crown of the plant (i.e., the canopy C) are separated from the roots R by the crop supports 36. The roots R hang free and are exposed to the ambient environment (air) of the growth chamber 44a in the feed zone F.

When needed, water (moisture) and nutrients are delivered directly (and in most cases exclusively) to the crop's dangling roots R and lower stem that extend below the crop supports 36 via an atomized or sprayed nutrient-rich water solution. The rest of the plant (e.g., it leaves, etc.) preferably remains relatively dry.

Preferably, the plants P (and/or other agricultural crops) are fed with water and nutrients during particular feeding intervals (which are associated with corresponding crop growth cycles). The crops may also be fed during intermediate intervals (e.g., intermediate rest intervals). That is, each pair of adjacent feeding intervals is preferably separated by an intermediate interval. Preferably, the plants P (and/or other crops) are fed during intermediate rest intervals, but do not receive light or cooling air. The plants P may be fed using a modified feeding schedule or varied nutrient levels during the intermediate intervals.

The plants P (and/or other agricultural crops) are fed during feeding intervals that each preferably extend about one and one-half (1.5) revolution about the spiral path 64. Each rest interval preferably extends about one-half (0.5) of a revolution about the spiral path 64. Thus, the ratio of feeding interval to rest interval is preferably about 3:1. In other preferred embodiments, the ratio of feeding interval to rest interval could range from about 1:1 to about 5:1.

Again, the train 74 and plants P preferably travel through about two (2) revolutions of the track 56 per day. In this manner, the system 30 is configured to expose the plants P to a generally circadian rhythm of feeding. However, it is within the ambit of the present invention where the cart 77 is advanced at a slower or faster speed along the track 56.

Because a continuous pattern of nozzles 144,160 is provided, the feeding and rest intervals are preferably provided by selectively turning nozzles on and off along the assembly length 54. Also, the feeding and rest intervals could have alternative lengths and/or configurations without departing from the scope of the present invention.

Depending upon the nozzle or mechanism used to generate the spray, the water solution delivered to the roots may be the form of macro water droplets, micro-droplets of mist, or even smaller fog droplets. In one or more embodiments, a hydro-atomized mist of about five (5) μm to about fifty (50) μm micro-droplets is preferred.

### Lighting System

Turning to FIGS. 5, 9, 14, and 14A, the lighting system 60 extends along the spiral path 64 and provides light to the plants P (and/or other agricultural crops) as the crops are advanced downwardly along the assembly length 54. The track 56 and the lighting system 60 cooperatively define the lighting zone L therebetween. The lighting system 60 is configured to illuminate the lighting zone L and thereby facilitate plant photosynthesis as the plants P are advanced along the spiral path 64.

The depicted lighting system 60 includes a continuous pattern of long and short lights 170a,b spaced along the assembly length 54 and secured to a metal framework 171 (see FIG. 9). The lights 170a,b are generally positioned above the track 56 at respective locations along the assembly length 54 by attaching the framework 171 relative to the supports 135. The illustrated lights 170a,b extend radially inwardly from adjacent the outer margin 68 of the spiral growing assembly 32 (see FIG. 9).

In other preferred embodiments, the lighting system includes a continuous pattern of only the depicted long lights 170a spaced along the assembly length 54 and secured to the framework (i.e., where the lighting system does not include the short lights). In these preferred embodiments, each of the long lights preferably provides a light intensity that progressively increases from a radially inner end of the light to a radially outer end of the light. In this manner, the arrangement of long lights can cooperatively provide a generally consistent light intensity from the inner margin 66 to the outer margin 68.

To the extent that the lighting system provides relatively greater light intensity along the inner margin than along the outer margin, it will be understood that different crops can be positioned along the respective inner and outer areas to best utilize the different light intensity values.

The long lights 170a preferably span substantially the full width of the spiral path 64, while the short lights 170b preferably span about two-thirds of the width of the spiral path 64 (see FIG. 9).

The lights 170a,b preferably comprise LED lights, although other types of lights could be used without departing from the scope of the present invention. Each of the depicted lights 170a,b preferably extends radially across the spiral path 64.

The illustrated lights 170a,b are also positioned in an alternating arrangement with each pair of long lights 170a having a short light 170b positioned therebetween (see FIG. 9). In this manner, the lights 170a,b are preferably configured and arranged to provide relatively higher light intensity in the area along the outer margin 68 (see FIG. 6) compared to the area along the inner margin 66 (see FIG. 6). However, it is also within the ambit of the present invention where one or more lights are alternatively configured to provide suitable light intensity for crop growth. Again, in other preferred embodiments, the system may not have an alternating light arrangement (e.g., where the system includes only long lights).

The lights 170a,b are also preferably configured to provide an adjustable light spectrum. That is, the lights 170a,b are preferably adjustable to change the spectrum of light emitted into the lighting zone L. In various embodiments, the lights may provide a customized radiant flux distribution. It will be understood that the light spectrum may be adjusted according to a particular phase of crop growth, which may be determined based upon the location of carts within the system (e.g., the vertical location of carts along the silo). The light spectrum may also be adjusted to replicate natural ambient light conditions or to otherwise provide desirable lighting conditions to maximize crop growth at different phases of crop growth.

Lighting provided by the depicted system 60 is preferably restricted to the lighting zone L at locations along the assembly length 54 while the train 74 and plants P are positioned in those locations (see FIG. 14). In particular, the illustrated bedway 118 is preferably opaque and spans the spiral path 64 to restrict light from passing upwardly from the lighting zone L to the adjacent feed zone F. Additionally, the carts 76, including the cap 82, are preferably opaque and spans the spiral path 64 to restrict light from passing downwardly from the lighting zone L, through the cart 76, and into the adjacent feed zone F.

In operation, the lighting system 60 is configured so that trains 74 support and advance plants P (and/or other agricultural crops) along the assembly length 54 while the crops are illuminated with light from the lighting system 60 during predetermined lighting intervals.

The plants P (or other agricultural crops) are preferably provided with light during particular lighting intervals (which are associated with corresponding crop growth cycles) and are generally not provided with light during intermediate rest intervals. That is, each pair of adjacent lighting intervals is preferably separated by an intermediate rest interval. More specifically, the plants P are fed during lighting intervals that each preferably extend about one and one-half (1.5) revolution about the spiral path 64. Each rest interval preferably extends about one-half (0.5) of a revolution about the spiral path 64. Thus, the ratio of lighting interval to rest interval is preferably about 3:1. In other preferred embodiments, the ratio of lighting interval to rest interval could range from about 1:1 to about 5:1. Most preferably, the lighting intervals are generally aligned with the feeding intervals.

As noted above, the train 74 and plants P preferably travel through about two (2) revolutions of the track 56 per day, with the plants P exposed to a generally circadian rhythm of light and dark cycles. Again, the cart 77 can also be advanced at a slower or faster speed along the track 56.

Because a continuous pattern of lights 170a,b is provided, the lighting and rest intervals are preferably provided by selectively turning lights on and off along the assembly length 54. However, the lights 170a,b could be configured so that the rest intervals along the assembly length 54 are devoid of lights 170. It is also within the scope of the present invention where the lighting and rest intervals have alternative lengths and/or configurations.

Because the illustrated lights are preferably secured in a fixed position at locations above the track 56, each respective crop support 36 moves along the spiral path 64, while the light source remains fixably mounted to provide periods of “light” and “dark” to the plants P (and/or other agricultural crops) as they move underneath. It will be appreciated that both the position of the lights and the speed of travel of the crop support structures can be varied depending upon the desired growth cycle for the crops. Many types of lighting may be utilized, specifically including, without limitation, growth cycle optimized spectrum LED lighting. Further, light intensity and spectrum may be altered level-to-level and/or crop-to-crop, as is the distance to the lights to correspond to the maturity of the crops.

The lighting system is preferably configured to provide light to the plants according to a desired Daily Light Integral (DLI). DLI provides a measure of cumulative photosynthetically active radiation (PAR) received by plants over the course of the day. It generally integrates light intensity in micro-mols per square meter per second (μmol/sq m-sec) and totals this over a 24-hour period (or a modified synthetic daylight period, such as 28 hours).

### Air System

Turning to FIGS. 7, 7A, 11, and 12, the air system 62 is configured to supply cooling air streams S to the growing spaces 46 associated with corresponding silo growth chambers 44a,b. The illustrated air system 62 includes multiple groups of air ducts 172a,b,c (see FIG. 7) and a supply system 174 (see FIG. 1) configured to generate cooling air and direct cooling air into the air ducts 172. Each air duct 172a,b,c preferably fluidly communicates with a respective air inlet opening 48 presented by the silo wall 42.

Again, the spiral segments 52, or spiral layers, extend a full revolution about the silo axis A1 and thereby provide one “level” of the spiral growing assembly 32. Each group of air ducts 172a,b,c is preferably associated with one of the spiral segments 52 so that the air ducts 172a,b,c communicate with the spiral segment 42 and provide cooling air thereto. Thus, the groups of air ducts 172a,b,c are preferably spaced along the silo axis A1.

Each spiral segment 52 preferably is associated with a corresponding group of air ducts 172a,b,c. However, it is also within the scope of the present invention where one or more spiral segments 52 are supplied with air by an alternative air duct configuration. For instance, in alternative embodiments, the air system could have an air duct that extends continuously along multiple spiral segments.

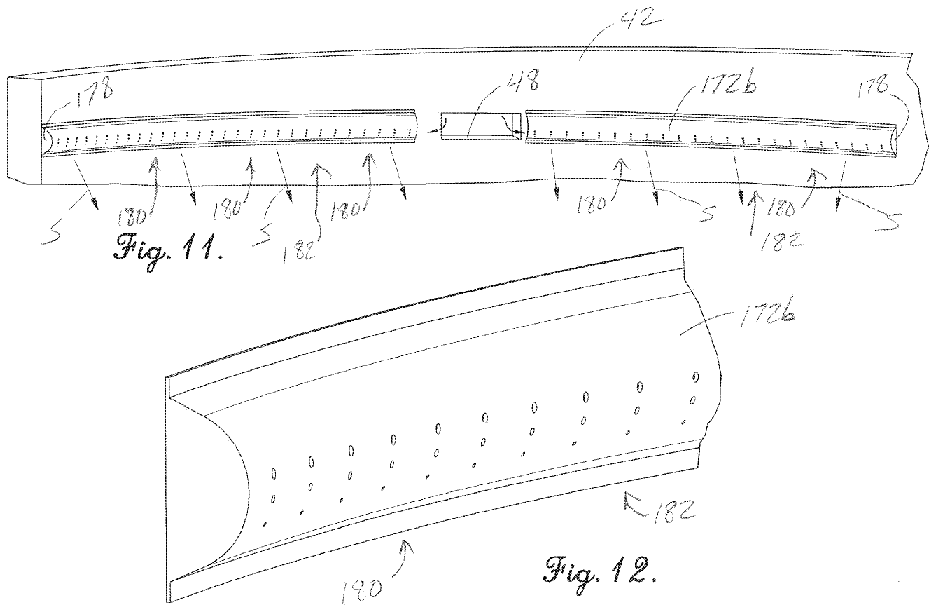

Each air duct 172a,b,c is mounted relative to a respective silo wall 42 and cooperates with the silo wall 42 to form a supply plenum 176 (see FIG. 7A). The air ducts 172a,b,c each present enclosed ends 178 and a pattern of duct openings 180 located between the ends 178 (see FIGS. 11 and 12). The duct openings 180 cooperatively provide an outlet 182 to discharge air into the growing space 46.

The air ducts 172a,b,c are preferably arranged and configured so that the air streams S are directed in a radially inward direction. The air ducts 172a,b,c also preferably facilitate air streams S that are generally uniform and have substantially the same air velocity.

As cooling air is directed into the spiral segments 52, the central access shaft T preferably receives warmer air (see FIG. 7A). The central access shaft T is generally open and unobstructed to permit warmer air to rise within the central access shaft T. In various embodiments, it will be appreciated that the air within the access shaft T can be externally vented to a location outside the silo growth chamber (e.g., an ambient location outside of the silo building 34). Air within the access shaft T can be externally vented with or without the use of a powered venting fan.

The air ducts 172a,b,c, are preferably designed to counteract frictional losses and pressure reductions along the air duct length in order to provide uniform air streams S with substantially uniform air velocity. For instance, the size and/or density of openings 180 can be progressively increased as the distance from the respective air inlet opening 48 increases. Although the air ducts 172 are depicted as having a generally constant cross-sectional duct size, it will be understood that the cross-sectional duct size can be reduced as the distance from the respective air inlet opening 48 increases in order to maintain air velocity.

The duct preferably comprises a formed sheet metal body that presents the openings 180. The duct 172 preferably includes a galvanized steel material. However, the duct 172 could include other materials, such as an alternative metal (e.g., stainless steel or aluminum) or a resin material (e.g., a plastic or synthetic resin material), without departing from the ambit of the present invention.

The duct 172 is preferably attached relative to the silo wall 42 and extends along the silo wall 42. The duct 172 is positioned in the silo growth chamber 44a,b and partly defines a radially outer margin of the growing space 46. When installed, each air duct 172a,b,c preferably communicates with a respective air inlet opening 48 presented by the silo wall 42.

Cooling air is preferably provided by the supply system 174, which includes a cooling tower 184 (see FIG. 1), supply fans 186 (see FIG. 7), compressor (not shown), condenser (not shown), and other HVAC equipment. The cooling air is preferably discharged into an interstitial bin 50 that serves as a silo air supply bin. The supply fans 186 force cooling air into respective bin chambers 50a,b of the interstitial bin 50, and cooling air is distributed among the air inlet openings 48 (see FIG. 7). Again, the bin chambers 50a,b each preferably receive cooling air but do not communicate with one another. The bin chamber 50a receives cooling air from one supply fan 186 and transmits cooling air to two of the silos 38. Each bin chamber 50b receives cooling air from a respective supply fan 186 and transmits the cooling air to a corresponding one of the silos 38.

In alternative embodiments, heating air may be required in some climates where the recycled waste heat from the LED lighting system is insufficient to warm the air to a hospitable temperature for the desired crop. It is also noted that external atmosphere may be used in place of HVAC equipment to provide atmospheric air to displace silo air when external temperatures and/or humidity are favorable to mechanical HVAC equipment.

Although one of the interstitial bins 50 is shown as receiving cooling air and supplying cooling air to the adjacent silos 38, it will be understood that one or more additional bins 50 in the silo building 34 can be supplied with cooling air and the adjacent silos 38 similarly configured to receive cooling air and distribute the cooling air using a similar configuration of air ducts 172.

The silo walls 42 preferably provide insulated walls that are suitable for transporting cooling air while restricting heat loss from the cooling air. However, an alternative ducting arrangement could be used for providing cooling air to the air inlet openings 48. In alternative embodiments, one or more ducts could be provided exterior to a silo 38 (e.g., in an interstitial bin 50) for supplying cooling air (e.g., to direct cooling air into air inlet openings 48 and/or to remove warm/hot air from the silo 38).

The plants P (and/or other agricultural crops) are preferably provided with cooling air during feeding intervals (which are associated with corresponding crop growth cycles) and are generally not provided with light during intermediate rest intervals. That is, each pair of adjacent lighting intervals is preferably separated by an intermediate rest interval. More specifically, the plants P are fed during lighting intervals that each preferably extend about one and one-half (1.5) revolution about the spiral path 64. Each rest interval preferably extends about one-half (0.5) of a revolution about the spiral path 64. Thus, the ratio of lighting interval to rest interval is preferably about 3:1. In other preferred embodiments, the ratio of lighting interval to rest interval could range from about 1:1 to about 5:1. Most preferably, the lighting intervals are generally aligned with the feeding intervals.

### Operation

In operation, the air system 62 supplies cooling air to bin chambers 50a,b of the bins 50 and the cooling air is further distributed into the air ducts 172 via the air inlet openings 48. The cooling air within the air ducts 172 is preferably discharged as groups of generally uniform cooling air streams S to various spiral segments 52 (see FIGS. 7 and 7A).

Again, as cooling air is directed into the spiral segments 52, the central access shaft T preferably receives warmer air. The central access shaft T preferably permits warmer air to rise within the central access shaft T and allows the warmer air to be externally vented.

In using the system 30 to grow batches of plants P (and/or other agricultural crops), the crops are placed into the crop openings 94 of the crop supports 36 adjacent the top of the spiral path 64. Trains 74 of carts 76,77 move downwardly along the track 56 and along the spiral path 64 at the predetermined speed. At desired feeding intervals, the roots of the crops can be misted, fogged, or otherwise provided with the required nutrient moisture. The excess nutrient-rich water is captured by the feeding system 58 and flows downward for capture and reuse if desired. At desired lighting intervals, the plant canopies are illuminated with a predetermined light intensity and spectrum. The growing space 46 is also provided with cooling air along the spiral path 64 as the train 74 of crops is advanced along the spiral path 64 in order to precisely control the air temperature and/or humidity experienced by the plants P.

The crops reach the end of their growth cycle at the bottom of the spiral path 64 where they can be harvested. As noted, a plurality of growth chambers can be grouped together and connected to take advantage of synergy among different types of crops. For example, composting heat from mushrooms M (or other fungi) in an adjacent chamber can be used in winter (or other periods of cold weather) to keep vegetables (or other crops) warm. Likewise, excess CO2 from mushrooms M (or other fungi) can be routed to adjacent chambers to increase crop growth rate. Additionally vegetable trimmings, root mass, spent mushroom substrate or other biological crop waste may be added to the top of a single silo to compost producing usable heat via hydronic distribution to other areas and finished compost at the bottom suitable for traditional or conventional soil-based farms.

The growing process with a fixed start and fixed end point provides a continuous growth gradient from top to bottom. Each layer of the spiral growing assembly can be optimized to the height of the crops corresponding to how many days the crops have been in the system. As the carts descend, the crops at each level will be a predictable height based on the velocity through the system and individual crop characteristics. The height of each level may therefore grow as the carts descend allowing lights to be an optimal distance from the crops. Additionally, the color spectrum of fixed lights may be altered to optimize growing conditions of each phase of life for the plants. Earlier starts may benefit from differing blue/red spectrum than mature crops at the bottom.

Natural temperature stratification may be used or manipulated to change temperature of varying levels or altitude within the facility. The coldest air is preferably at the harvest station where plants P will be cooled prior to refrigeration.

This innovative design allows for the same build to satisfy a vast array of crops, including, without limitation, vegetables or mushrooms. Specialty mushroom crops require sterilization or super pasteurization, inoculation with mushroom (or other fungi) spawn and periods of incubation to colonize a substrate. This is traditionally done in polypropylene or polyethylene bags with micropore breathable filters to exclude airborne contaminants. Blocks are encouraged to run the mycelium under warm conditions and after thorough colonization they are encouraged to be cooled prior to fruiting the blocks in a grow room. The innovative crop growing system described herein benefits the incubation process by providing a gradient of temperatures naturally through temperature stratification (heat rises). The same installed technology can be used to incubate mushrooms from top to bottom as with crops, described above, except without the need for the above-described misting/moisture/humidification and light systems (except as needed to service the system or maintain minimum ambient humidity or promote growth in some species that require light near the end of their incubation cycle).

| Vegetables | Mushrooms |
|---|---|
| Warm at top, cooler bottom | <same |
| Bright colored light, | No light, turn lights off |
| close to leaves | |
| Requires warmth in winter | Exothermic: Generates heat |
| Thrives with CO2 | Generates CO2 |
| | when incubating |
| Needs ~15″ clearance | Needs 18″ clearance |
| Needs fine droplet mist | No mist or fog required. |
| | Equipment not installed or unused. |

It will be appreciated that the present system 30 provides a number of advantages, including continuous, high-efficiency and high-throughput crop production. The system 30 also provides lower construction cost, with less capital per unit product and faster startup. The use of aeroponics/fogponics in the disclosed system 30 has been found to be generally more productive than aquaponics/hydroponics and traditional farming techniques.

The illustrated system 30 also provides a high-efficiency crop growth system that utilizes efficient crop movement and logistics to improve worker efficiency and worker safety while minimizing the number of workers. The system 30 also includes a variety of energy efficient features that conserve electrical and thermal energy and provide efficient environmental control. The system 30 further enables an efficient use of water and nutrients and provides an efficient cleaning in place (CIP) system while minimizing the risk of algae growth, pest infestation, other forms of contamination, including, without limitation, the presence of E. coli and other bacteria, and system cleaning downtime. The system 30 is also highly adaptable to accommodate a wide variety of crops, and the system 30 can be readily reconfigured to switch between different types of crops or other agricultural products.

These, and other, advantages of embodiments of the inventions will be more readily appreciated with reference to specifically contemplated embodiments. Although the above description presents features of preferred embodiments of the present invention, other preferred embodiments may also be created in keeping with the principles of the invention. Such other preferred embodiments may, for instance, be provided with features drawn from one or more of the embodiments described above. Yet further, such other preferred embodiments may include features from multiple embodiments described above, particularly where such features are compatible for use together despite having been presented independently as part of separate embodiments in the above description.

The preferred forms of the invention described above are to be used as illustration only, and should not be utilized in a limiting sense in interpreting the scope of the present invention.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination. The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

1. A silo growing system configured to grow an agricultural crop, said silo growing system comprising:
a silo presenting a vertically elongated silo growth chamber;
a spiral growing assembly positioned in the silo growth chamber and extending along an assembly length to at least partly define a spiral growing space to receive and feed the agricultural crop therein, said spiral growing assembly including a continuous track and a feeding system,
said track extending continuously along the assembly length of the spiral growing assembly and presenting a generally downward spiral path that defines a path axis, with the track configured to direct the agricultural crop along the spiral path,
said feeding system extending along the track to direct a supply of water and/or nutrients in the spiral growing space along the spiral path by providing direct root application of the supply of water and/or nutrients to the agricultural crop; and
a movable crop support configured to support at least some of the agricultural crop, said movable crop support operably supported by the track and configured to be advanced downwardly along the assembly length to thereby direct the agricultural crop through the growing space along the spiral path.

2. The silo growing system as claimed in claim 1,
said spiral growing assembly including a series of spiral segments arranged end-to-end, with each pair of adjacent spiral segments revolving about a silo axis and at least partly overlapping one another.

3. The silo growing system as claimed in claim 2,
said spiral growing assembly presenting an inner margin that defines an inner diameter dimension, with the inner diameter dimension being substantially constant along the length of the silo axis.

4. The silo growing system as claimed in claim 1, said movable crop support comprising a movable cart, said movable cart including a frame and multiple wheels rotatably attached relative to the frame, with the wheels operably engaged with the track and configured to roll along the track as the movable cart is advanced downwardly along the spiral path.

5. The silo growing system as claimed in claim 1, said track including a pair of rails positioned side-by-side and extending along the path axis.

6. The silo growing system as claimed in claim 5, said movable crop support comprising a movable cart including a frame and multiple wheels rotatably attached relative to the frame, with the wheels operably engaged with corresponding rails and configured to roll along the rails as the movable cart is advanced downwardly along the spiral path.

7. The silo growing system as claimed in claim 1, said movable crop support comprising a movable cart including a cap that presents multiple crop openings, with the openings configured to receive corresponding ones of the agricultural crop so that crop roots are positioned below the cap and crop leaves are positioned above the cap, wherein the cap is supported by the frame.

8.-9. (canceled)

10. The silo growing system as claimed in claim 7,
said feeding system cooperating with the track to define a feed zone therebetween, with the feeding system generally positioned below the track at locations along the assembly length,
said cap being opaque and spanning the path to restrict light from passing through the movable cart and into the feed zone,
said movable crop support comprising a movable cart including a mesh bottom that spans the frame to support the agricultural crop and permits roots of the crop to pass into the feed zone.

11. The silo growing system as claimed in claim 1, further comprising:
a plurality of movable crop supports including the first-mentioned movable crop support and being configured to support the agricultural crop,
said movable crop supports configured to be supported by the track and advanced downwardly along the assembly length to thereby direct the agricultural crop through the growing space along the spiral path,
said movable crop supports being arranged in series with each other when positioned on the track.

12. The silo growing system as claimed in claim 1,
said feeding system including a collection bedway that extends along the path and is generally positioned below the track at locations along the assembly length,
said track and said collection bedway cooperatively defining a feed zone therebetween, with the feed zone configured to at least partly receive the agricultural crop and permit application of the supply of water and/or nutrients to the agricultural crop inside the feed zone.

13. The silo growing system as claimed in claim 12,
said feeding system including dispensing nozzles to discharge the supply of water and/or nutrients inside the feed zone, with the tray configured to collect any excess part of the supply of water and/or nutrients,
said dispensing nozzles of the feeding system each including a sprayer, mister, and/or fogger.

14.-15. (canceled)

16. The silo growing system as claimed in claim 13,
said collection bedway including a bottom wall and opposite sidewalls that extend upwardly from the bottom wall, with the bottom wall and sidewalls cooperatively defining a channel,
said track including a pair of rails positioned side-by-side and extending along the path axis, said sidewalls being attached relative to respective ones of the rails,
said sidewalls presenting an upper margin of the collection bedway, with the upper margin and floor defining a channel height dimension, said sidewalls being flexible to permit the channel height dimension to increase and/or decrease so as to accommodate crop root depth.

17.-18. (canceled)

19. The silo growing system as claimed in claim 1,
said feeding system at least partly defining a feed zone configured to at least partly receive the agricultural crop and permit application of the supply of water and/or nutrients to the agricultural crop inside the feed zone,
said feeding system including dispensing nozzles to discharge the supply of water and/or nutrients to the agricultural crop inside the feed zone.

20. (canceled)

21. The silo growing system as claimed in claim 1,
said spiral growing assembly including a lighting system that extends along the path,
said track and said lighting system cooperatively defining a lighting zone therebetween, with the lighting system configured to illuminate the lighting zone and thereby facilitate crop photosynthesis as the agricultural crop is advanced along the spiral path.

22. The silo growing system as claimed in claim 21,
said lighting system including a series of lights spaced along the assembly length and being generally positioned above the track at locations along the assembly length,
at least some of said series of lights extending radially across the spiral path,
said feeding system including a collection bedway that extends along the path and is generally positioned below the track at locations along the assembly length, said collection bedway being opaque and spanning the path to restrict light from passing from the lighting zone to the feed zone.

23. The silo growing system as claimed claim 1,
said spiral growing assembly including an air system configured to supply air to the growing space,
said air system including a duct that at least partly defines a supply plenum,
said duct extending along the assembly length and presenting an outlet to discharge the air into the growing space.

24. The silo growing system as claimed in claim 23,
said duct being positioned in the silo growth chamber and at least partly defining a radially outer margin of the growing space,
said silo including a silo wall that presents the silo growth chamber and partly defines the growing space, with the duct being attached relative to the silo wall, and/or
said air system including a series of ducts that each at least partly define respective supply plenums, said ducts extending along the assembly length and presenting outlets to discharge the air into the growing space.

25.-26. (canceled)

27. The silo growing system as claimed in claim 1,
said silo presenting a plurality of vertically elongated silo growth chambers including the first-mentioned silo growth chamber, said silo growth chambers optionally being in fluid communication with one another,
said silo growth chambers extending vertically alongside one another; and
a plurality of spiral growing assemblies including the first-mentioned spiral growing assembly, each of said spiral growing assemblies positioned in a respective one of the silo growth chambers.

28. (canceled)

29. The silo growing system as claimed in claim 27, further comprising: a fungi production assembly positioned in a respective one of the silo growth chambers and configured to incubate fungi therein, said silo growth chambers being in fluid communication with one another, with heat and carbon dioxide from the fungi in the respective silo growth chamber configured to be transmitted to the agricultural crop in the other silo growth chambers.

30. The silo growing system as claimed in claim 27, said silo presenting a silo air supply bin extending vertically alongside the silo growth chambers and fluidly communicating with each of the silo growth chambers.

31. A spiral growing system configured to be housed in a vertically elongated silo growth chamber to grow an agricultural crop, said spiral growing system comprising:
a spiral growing assembly extending along an assembly length to at least partly define a spiral growing space to receive and feed the agricultural crop therein, said spiral growing assembly including a continuous track and a feeding system,
said track extending continuously along the assembly length of the spiral growing assembly and presenting a generally downward spiral path that defines a path axis, with the track configured to direct the agricultural crop along the spiral path;
said feeding system extending along the track to direct a supply of water and/or nutrients in the growing space along the spiral path by providing direct root application of the supply of water and/or nutrients to the agricultural crop; and
a movable crop support configured to support at least some of the agricultural crop, said movable crop support operably supported by the track and configured to be advanced downwardly along the assembly length to thereby direct the agricultural crop through the growing space along the spiral path.

32.-52. (canceled)

53. A method of growing an agricultural crop using aeroponics, fogponics, and/or nutrient film technique, said method comprising the steps of:
(a) positioning agricultural crop on a spiral path;
(b) facilitating advancement of the agricultural crop downwardly along the spiral path;
(c) providing direct root application of water and/or nutrients to the agricultural crop to grow the agricultural crop as the agricultural crop is advanced along the spiral path; and
(d) harvesting the agricultural crop from the spiral path.

54. The method as claimed in claim 53,
step (a) including the steps of supporting the agricultural crop on a movable cart and positioning the movable cart on the spiral path,
step (b) including the step of advancing the movable cart and agricultural crop together along the spiral path.

55. The method as claimed in claim 53,
step (a) including the steps of supporting the agricultural crop on a plurality of movable carts, arranging the movable carts in series with one another, and positioning the movable carts on the spiral path,
step (b) including the step of advancing the movable carts and agricultural crop together along the spiral path.

56. The method as claimed in claim 55, further comprising the step of:
(e) braking a leading one of the movable carts to control advancement of the movable carts during step (b).

57. The method as claimed in claim 53,
step (c) including the step of providing a supply of water and nutrients to the agricultural crop by dispensing the solution as a mist and/or fog.
